(19) United States
(12) Patent Application Publication
(10) Pub. No.: US 2025/0314651 A1
(43) Pub. Date: Oct. 9, 2025

(54) PERMEABLE BIOELECTRONIC SYSTEMS AND METHODS FOR MAKING THE SAME

(71) Applicant: THE HONG KONG POLYTECHNIC UNIVERSITY, Hong Kong (CN)

(72) Inventors: Qiuna ZHUANG, Hong Kong (CN); Zijian ZHENG, Hong Kong (CN)

(21) Appl. No.: 18/627,693

(22) Filed: Apr. 5, 2024

(51) Int. Cl. G01N 33/543; G01N 35/00; H05K 1/03; H05K 1/11

(52) U.S. Cl. CPC G01N 33/5438; H05K 1/0393; H05K 1/113; G01N 2035/00366

(57) ABSTRACT

A permeable bioelectronic system comprising stretchable multilayered circuits comprising liquid metal (LM), and LM interconnects for bonding one or more inorganic electronic circuit components to the stretchable multilayered circuits.

### FIELD OF THE DISCLOSURE

The present disclosure generally relates to permeable bioelectronic electronics.

### BACKGROUND

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Permeable, soft, and stretchable integrated electronic systems possessing continuous sensing and intervention abilities and wearing/implanting comfort are essential for a broad range of emerging applications, such as intensive care, rehabilitation, close-loop diagnosis/treatment, and virtual reality/augmented reality. In the past two decades, enormous progress has been made in developing novel materials and architectures for stretchable electronics. In particular, structural approaches based on lateral strain-tolerant island-bridge engineering (buckle, serpentine, spring) and vertical strain-isolation engineering (thickness, stiffness, and elasticity) offer remarkable tools to integrate conventional rigid integrated circuit (IC) components (transistors, capacitors, resistors, sensors, communication, and energy parts, etc.) with highly stretchable polymeric substrates to generate a form of stretchable hybrid electronics, which not only take advantages of the mature IC design and manufacture but also meet the mechanics of soft organs and tissues.

It is an object of the present disclosure to overcome or substantially ameliorate one or more of the disadvantages of prior art, or at least to provide a useful alternative.

### SUMMARY

According to one or more embodiments, there is provided a permeable bioelectronic system comprising stretchable multilayered circuits comprising liquid metal (LM), and LM interconnects for bonding one or more inorganic electronic circuit components to the stretchable multilayered circuits.

According to one or more embodiments, there is provided a method for making a permeable bioelectronic system, the method comprising: generating a microcircuit having a first side and a second side; transferring the microcircuit onto a fiber mat such that the first side of the microcircuit contacts the fiber mat; forming a paste mask layer onto the second side of the microcircuit; forming a base circuit layer onto a side of the fiber mat, the side being away from the first side of the microcircuit, the base circuit layer comprising liquid metal (LM); forming stretchable vertical interconnect accesses (VIAs) for electrically connecting the microcircuit, the base circuit layer and the paste mask layer, the VIAs being filled with LM; forming hybrid LM (hLM) solder onto the paste mask layer for bonding one or more inorganic electronic circuit components; and forming an encapsulation layer for encapsulating the one or more inorganic electronic circuit components, the paste mask layer, the base circuit layer and the microcircuit.

Other example embodiments are discussed herein.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The drawings are not to scale, unless otherwise disclosed. Certain parts of the drawings may be exaggerated for explanation purposes and shall not be considered limiting unless otherwise specified.

FIG. 1A illustrates an exploded view of a permeable three-dimensional integrated electronic skin (P3D-eskin) according to certain embodiments of the present disclosure. Liquid metal (LM) microelectrodes are adopted as reliable interfaces between the soft, rough fiber mat substrate and rigid components. Vertical interconnect accesses (VIAs) are used for interlayer electrical connections. Electronic circuit components in each layer may include a microcontroller unit (MCU), an oscillator, a multiplexer (MUX), a current mirror, a digital-analog-convertor (DAC), an operational amplifier (OP-AMP), a high voltage module (HV, 20V), and a low dropout regulator (LDO, 3.3 V). The dashed lines indicate the distribution and positions of the VIAs in the system.

FIG. 1B depicts a digital image of permeable 3D LM circuits and a partially oxidized LM (oLM) pad.

FIG. 1C depicts a digital image of the soft and stretchable P3D-eskin with hybrid LM (hLM) solder.

FIG. 1D depicts a digital image demonstrating the stable electrical performance of the bent (550% strain) P3D-eskin.

FIG. 1E depicts a digital image demonstrating the stable electrical performance of the stretched (550% strain) P3D-eskin.

FIG. 1F illustrates the permeability to air and moisture of P3D-eskin.

FIG. 1G depicts air and moisture permeabilities of several wearable substrates including P3D-eskin, PDMS-eskin, wound dressing, medical tape, and cotton fabric according to certain embodiments. Each type of substrate corresponds to a pair of columns, where the left column represents moisture permeability, and the right column represents air permeability. The error bars stand for the standard deviation (SD) and the bar height represents mean value.

FIG. 1H depicts digital images showing (a) the skins are covered by P3D-eskin (upper) and PDMS-eskin (lower) respectively and (b) the skin status after attaching P3D-eskin (upper) and PDMS-eskin (lower) for one week. The skin area covered by the P3D-eskin is inflammation-free while the skin area covered by the PDMS-eskin displays serious skin erythema.

FIG. 1I depicts a circuit design of a P3D-eskin system using Bluetooth technology according to certain embodiments. The electronic circuit components in each layer include a MCU, an oscillator, a MUX, a current mirror, a DAC, an OP-AMP, a HV (20V), and a LDO (3.3 V).

FIG. 2A depicts a processing flow of layer-by-layer fabrication of P3D-eskins according to certain embodiments of the present disclosure.

FIG. 2B depicts digital images showing the structure and functions of each layer in P3D-eskins: (a) upper layer of LM 3D circuits; (b) base layer of LM 3D circuits; (c) oLM paste mask layer; (d) electronic components; (e) permeable superstrate.

FIG. 3A depicts digital images showing stencil printing of complex high-density patterns in the upper circuit layer and coil antenna according to certain embodiments of the present disclosure: (a) laser-cutting mask for the upper layer circuit; and (b) laser-cutting mask for coil antenna. For complex and high-density patterns in the upper circuit layer and an antenna coil, stencil printing cannot serve as a reliable technique, which is very likely to make the mask be detached and thus ruins the pattern.

FIG. 3B depicts digital images showing stencil printing of simple patterns in the base layer and paste mask layer of LM 3D circuits according to certain embodiments of the present disclosure. It is shown that stencil printing works well for these simple patterns.

FIG. 4A depicts a printed circuit board (PCB) fabrication compatible laser cutting machine (LPKF U4) according to certain embodiments of the present disclosure.

FIG. 4B depicts the fixing frame with markers for stencil printing.

FIG. 4C depicts the resolution and trace density of the modified stencil printing with patternable linewidths: (a) 500 μm line without gap; (b) 200 μm line without gap; (c) 100 μm line and gap with a successful rate of ˜70% (that is, around 70% of the line arrays can be successfully patterned by the stencil printing technique); and (d) 50 μm line with gaps.

FIG. 5 depicts scanning electron microscope (SEM) and elemental mapping images showing (a) the selective wetting of micropatterned Ag and LM with (b) the formation of AgIn alloy according to certain embodiments of the present disclosure. The selective wetting of LM lies in the contrast between the LM-lyophobic property of the poly(styrene-block-butadiene-block-styrene) (SBS) mat and the LM-lyophilic property of Ag. Therefore, in the fabrication of the LM microcircuit, EGaIn wets only the Ag-covered areas and dewets from the SBS surface. When applying EGaIn on Ag, reactive alloying between Ag and In forms AgIn alloys. Additional EGaIn will subsequently wet the AgIn alloy layer, and form the EGaIn/AgIn/Ag trilayer, showing the stronger signal of Ga and the weaker signal of Ag (deeper detection distance) in the elemental mapping.

FIG. 6 depicts digital images of (a) battery-powered P3D-eskin equipped with Bluetooth, (b) the battery-powered P3D-eskin worn on a user's arm, and (c) a battery-free type of P3D-eskin on an NFC reader according to certain embodiments of the present disclosure.

FIG. 7 depicts (a) the air/moisture-permeable but waterproof P3D-eskin system, (b) the mechanism, and (c) contact angle (CA) of water on the monolithic and hydrophobic P3D-eskin system according to certain embodiments of the present disclosure. The microporous fibrous structure of the electrospun fiber mat allows air molecules and moisture (water vapor) to pass through it. Meanwhile, the SBS fiber mat is intrinsically hydrophobic with a large water contact angle, thereby preventing liquid-state substances from penetrating through the whole electronic system.

FIG. 8 depicts rain test of P3D-eskin system according to certain embodiments of the present disclosure, where (a) a schematic illustration of the setup of rain test, (b) a digital image of the setup of rain test, (c1) digital images showing the waterproofness of the P3D-eskin system with a hydrophobic surface, (c2) surface of waterproof P3D-eskin system, and (d1) digital images showing the blotting paper is not waterproof that no observable water on the blotting paper, and (d2) surface of the blotting paper.

FIG. 9 depicts digital images showing the electrical stability of the P3D-eskin system with stable LED luminance in (a) water and (b) artificial sweat (pH value: 4.7±0.1) and (c) artificial sweat (pH value: 4.7±0.1).

FIG. 10A depicts digital images showing the overall thickness of the PDMS-eskin according to certain embodiments of the present disclosure.

FIG. 10B depicts digital images showing the overall thickness of the P3D-eskin according to certain embodiments of the present disclosure. For the fabrication of P3D-ekins, all layers including the substrate, interlayers, and encapsulation layer are made of soft, porous and super-elastic fiber mats. The electrospun encapsulating fiber mat shows conformal contact with the underlying rigid components, which is distributed along the topography of the components and largely alleviates the system bulkiness.

FIG. 10C depicts stress-strain curves of PDMS-eskins of FIG. 10A and the P3D-eskins of FIG. 10B.

FIG. 10D depicts modulus values of PDMS-eskins of FIG. 10A and the P3D-eskins of FIG. 10B. The PDMS-eskins are thicker by ˜54% and more rigid by ˜60% due to the compact and thin-film type of layout following the conventional spin-coating and casting process.

FIG. 11A depicts SEM images of LM/SBS with various heating durations before and after stretch according to certain embodiments of the present disclosure. The OLM is prepared by oxidizing LM in the air. After heating LM with increasing duration ranging from 0 to 24 h, the size of the gallium oxide increases from several μm to several hundred μm. After being pre-stretched under 1500% strain for 12 cycles, the continuous thin film (heating duration less than 16 h) self-organizes into a laterally mesh-like and vertically buckled structure, with the formation of nodes from the strong oxide layer.

FIG. 11B depicts XPS results of oLM after various heating durations. Ga 2p (3/2) spectrum shows a predominant peak with a binding energy of 1118.8 eV from Ga₂O₃, with the presence of Ga metal (1116.5 eV) and Ga₂O (1118.2 eV).

FIG. 11C depicts formation of the gallium oxides (Ga₂O₃ and Ga₂O) during the heating of LM. With increasing heating duration, the signals of Ga₂O₃ and Ga₂O become stronger.

FIG. 11D depicts Young's modulus of oLM/SBS after various heating durations. The error bars represent standard deviation (SD) and the scatter values represent mean values. Due to the strong oxidation of LM, the average modulus of oLM/SBS increases from ˜0.1 MPa (9841 Pa) with the heating duration of 0 h (i.e., LM/SBS) to ˜0.31 MPa with the heating duration of 24 h. Accordingly, the stiffness is also enhanced by around 2 folds while the thickness remains unchanged.

FIG. 11E depicts electrical conductivity of oLM/SBS after various heating durations. The OLM/SBS with a heating duration of 16 h maintains a high electrical conductivity of over 28,300 S/cm. The error bars represent SD and the scatter values represent mean values.

FIG. 11F depicts resistance changes of hybrid LMs (hLM) (weight ratio of oLM and LM and =1:2, oLM with various heating durations) on the SBS fiber mats as the function of tensile strain.

FIG. 12 depicts wetting properties of (a) pristine LM on the fiber mat and (b) partially oxidized LM (oLM) on the fiber mat with the same loading amount (20 mg) in air. In the oxidative environment (in the air), the droplet of pristine LM adopts a metastable and nonspherical shape on the fiber mat. After the oxidation treatment, oLM is of much high wettability to the fiber mat with the formation of a super lyophilic surface coating.

FIG. 13A depicts schematic illustrations and scanning electron microscope images showing the electrical interfaces of rigid components using pristine (scale bar=200 μm) according to certain embodiments of the present disclosure.

FIG. 13B depicts schematic illustrations and scanning electron microscope images showing the electrical interfaces of rigid components using oLM according to certain embodiments of the present disclosure (scale bar=200 μm) according to certain embodiments of the present disclosure.

FIG. 13C depicts schematic illustrations and scanning electron microscope images showing the electrical interfaces of rigid components using hLM solder (scale bar=200 μm) according to certain embodiments of the present disclosure.

FIG. 13D depicts the finite element analysis (FEA) of stress distribution of the electrical interface using ultrastretchable hLM solder.

FIG. 13E depicts electrical resistance of the electrical interfaces of microresistors (0603, ˜100Ω) using pristine LM, oLM, and hLM solder respectively, where “0603” refers to the fingerprint of the electronic components.

FIG. 13F illustrates 3D electrical connection and interfaces between the rigid IC and the ultrastretchable hLM solder. oLM serves as contact pads, while pristine LM serves as patterned in-plane interconnects, VIAs, and additional contact paste.

FIG. 13G depicts cross-sectional SEM images showing a rigid microchip integrated with the 3D LM circuit at zero strain.

FIG. 13H depicts cross-sectional SEM images showing a rigid microchip integrated with the 3D LM circuit at 50% strain. The interfaces between the rigid chip and the LM circuit are well maintained under the large tensile strain, and the LM circuit is stretched in the 3D space.

FIG. 13I depicts electrical resistances of a series of highly stretchable microresistor-integrated 3D LM circuits at zero strain.

FIG. 13J depicts electrical resistances of a series of highly stretchable microresistor-integrated 3D LM circuits at 1500% strain for 1000 cycles. The electrical resistances show outstanding stability when the circuits are stretched to 1500% for 1000 cycles.

FIG. 13K depicts transfer characteristics of stretchable P-type metal-oxide-semiconductor field effect transistors (MOSFETs).

FIG. 13L depicts transfer characteristics of stretchable N-type MOSFETs.

FIG. 13M is digital images of a stretchable logic circuit (clock-controlled switch) fabricated with the stretchable MOSFETs, where it shows the circuit is connected with flexible printed circuit board (FPCB) and encapsulated with permeable super-elastic fiber mat.

FIG. 13N depicts logic outputs of stretchable logic circuit (clock-controlled switch) fabricated with the stretchable MOSFETs.

FIG. 14 depicts FEA of the stress distribution of the electrical interface using single-component LM solder according to certain embodiments of the present disclosure. It shows the FEA results of stress distribution of the connection interface between a rigid microchip and the single-component LM solder at 50% tensile strain. The maximum stress occurs on the interface between the rigid microchip and the single-component LM solder. The stress concentration factor (the ratio of the maximum stress to the average stress, i.e., σₘₐₓ/σ_{avg}) at the interface between the rigid chip and the soft fiber mat was 30% larger than using hybrid LM (hLM) solder.

FIG. 15 depicts a digital image showing the stable performance of a lighted light-emitting diode (LED) using hLM solder (a) before and (b) after stretch. Conventionally, pristine LM is difficult to be directly applied on various solid surfaces due to its intrinsic high surface tension. Here the oLM pads are multi-wettable to the pins of the component, pristine fluidic LM, and the fiber mat, which ensures electrically stable connections both in-plane and out-of-plane. Further, with the encapsulation of a thin permeable super-elastic fiber mat, the electronic components can be immobilized onto the hLM solder, leading to better electrical stability under a large strain.

FIG. 16A depicts output characteristics of the stretchable P-Channel enhancement mode MOSFETs using hLM as an electrical interface at 0% strain according to certain embodiments of the present disclosure.

FIG. 16B depicts output characteristics of the stretchable P-Channel enhancement mode MOSFETs using hLM as an electrical interface at 500% strain according to certain embodiments of the present disclosure.

FIG. 16C depicts output characteristics of the stretchable N-Channel enhancement mode MOSFETs using hLM as an electrical interface at 0% strain according to certain embodiments of the present disclosure.

FIG. 16D depicts output characteristics of the stretchable N-Channel enhancement mode MOSFETs using hLM as an electrical interface at 500% strain according to certain embodiments of the present disclosure.

FIG. 17A depicts design of the permeable stretchable logic circuits including (a) inverse gate, (b) NOR gate, and (c) clock-controlled switch according to certain embodiments of the present disclosure.

FIG. 17B depicts outputs of the logics validated with the rigid printed circuit boards: (a) inverse gate; (b) NOR gate; and (c) Switch.

FIG. 17C depicts a digital image of the inverse gate of FIG. 17A.

FIG. 17D depicts a digital image of the NOR gate of FIG. 17A.

FIG. 17E depicts logic outputs of the inverse gate of FIG. 17A.

FIG. 17F depicts logic outputs of the NOR gate of FIG. 17A.

FIG. 17G illustrates a permeable 3D integrated stretchable switch array according to certain embodiments of the present disclosure.

FIG. 17H depicts the threshold driving voltage of the switch array at a strain of 100%.

FIG. 17I depicts a statistic analysis of the transconductance of the 64-channel switch array. The switches are used for controlling loads and in complementary metal-oxide semiconductor (CMOS) digital circuits as they operate between their cut-off and saturation regions. The multi-channel switch array depicts a uniform threshold driving voltage (Vg) of ˜1.75 V at a strain of 50%, and an average transconductance of ˜100 mS.

FIG. 17J depicts a digital image of the permeable 3D integrated stretchable switch array at 100% strain.

FIG. 18A depicts electrical resistance changes of LM circuit traces (200 μm linewidths, with and without storage in air for 8 months) during stretch-release cycling tests of 100% strain.

FIG. 18B depicts electrical resistances of a series of highly stretchable microresistor-integrated 3D LM circuits that are stored in the air for 8 months.

FIG. 19A depicts SEM images showing Mode 1 of mechanical failure: long-term continuous mechanical wearing/tearing of the stress-concentrated rigid-soft interface in a cycle-dependent manner according to certain embodiments of the present disclosure. This case occurs when the solder joints/vias sustains extremely large amounts of repeated loading-unloading process at low strains (e.g., 100% strain for over 10000 cycles).

FIG. 19B depicts SEM images showing Mode 2 of mechanical failure: the fracture of the substrate material according to certain embodiments of the present disclosure. When the solder joints/vias sustains repeats loading-unloading process at high strains (e.g., 1500% strain for over 1000 cycles), the substrate material fractures while the solder joints are still well encapsulated by the superstrate fiber mat. Nevertheless, the whole system fails in this case as well.

FIG. 19C depicts FEA of the stress distribution of the electrical interface with soft and super-elastic encapsulation mat. The maximum stress occurs on the rigid-soft interface between the rigid microchip and the soft SBS fiber mat.

FIG. 19D depicts tensile force of the SBS fiber mats (n=10) as the function of extension.

FIG. 20A depicts digital images showing no leakage of LM residue on the skin under various pressing loads: (a) pressure: 0 kPa, (b) pressure: 12.5 kPa, (c) pressure: 25 kPa, and (d) pressure: 50 kPa.

FIG. 20B depicts digital images showing intact pattern of LM 3D circuit without LM leakage onto the skin during the pressing test: (a) pristine LM 3D circuit before mounting components, (b) pressing the LM 3D circuit onto the skin, (c) skin indentation appears without LM leakage, and (d) intact LM 3D circuit after pressing test.

FIG. 21 depicts digital images of an overstretching P3D-eskin system encapsulated with a permeable super-elastic fiber mat without the leakage of LM.

FIG. 22A depicts a block diagram of a sensing system and a customized mobile app according to certain embodiments of the present disclosure. Electrical stimulations can be delivered to the bicep femoris on a rat, and the corresponding electromyography (EMG) signals are recorded using the LM microelectrode.

FIG. 22B is a digital image showing the long-range wireless communication of the sensing system at a distance of 15 m.

FIG. 22C depicts generated stimulation pulses with controlled duty cycle ranging from 1% to 10%, frequency fixed at 100 Hz.

FIG. 22D depicts generated stimulation pulses with controlled repetition frequencies ranging from 5 Hz to 100 Hz at the dry state.

FIG. 22E depicts generated stimulation pulses with controlled repetition frequencies ranging from 5 Hz to 100 Hz at the steamed state.

FIG. 22F depicts digital images showing the P3D-eskin is steamed on top of boiling water.

FIG. 22G depicts generated stimulation current pulses with controlled current intensity under different wirelessly sent commands (0x40 to 0x60) at the dry and the steamed state (fixed load: 1 kΩ).

FIG. 22H depicts a digital image of the wireless transcutaneous electrostimulation and electrophysiological sensing system based on the P3D-eskin platform. The system is attached to the bicep femoris of a rat.

FIG. 22I depicts evoked EMG response signals under stimulation frequencies of (a) 1 Hz, (b) 5 Hz, and (c) 10 Hz respectively.

FIG. 22J depicts a spectrogram of the EMG signals in response to the electrostimulation input generated by the P3D-eskin at a frequency of 5 Hz.

FIG. 23A depicts a digital image showing the impermeability of the PDMS-eskin. When the entire PDMS-eskin is steamed on top of boiling water, numerous water droplets accumulate on the surface of the PDMS-eskin because of its poor permeability.

FIG. 23B is a partially enlarged view of FIG. 23A.

FIG. 24 depicts a schematic illustration of the current control module of the P3D-eskin system for wireless transcutaneous electrostimulations and electrophysiological sensing functions. The current control module comprises two basic parts including the control part and the monitoring part. In the control part, the intensity of the controlled stimulation current (ICTL) is identical to the one of the reference currents owing to the current mirror circuit. The reference current intensity is determined by the DAC output signal voltage amplitude controlled by the MCU. In the monitoring part, the current mirror is connected in series with a fixed resistance (50Ω) before the ground, where the voltage is linearly related to the actual current intensity (ICTL=V/100). The sensed voltage is duplicated by a voltage follower before inputting into the ADC on MCU, to prevent sudden overload.

FIG. 25A depicts current characteristics vs time for the wireless electrostimulation output of a P3D-eskin system at 0% stretching state according to certain embodiments of the present disclosure.

FIG. 25B depicts current characteristics vs time for the wireless electrostimulation output of a P3D-eskin system at 20% stretching state according to certain embodiments of the present disclosure.

FIG. 25C depicts current characteristics vs time for the wireless electrostimulation output of a P3D-eskin system at 50% stretching state according to certain embodiments of the present disclosure.

FIG. 25D depicts current characteristics vs time for the wireless electrostimulation output of a P3D-eskin system at 100% stretching state according to certain embodiments of the present disclosure.

FIG. 25E depicts voltage characteristics vs time for the wireless electrostimulation output of a P3D-eskin system at 0% stretching state according to certain embodiments of the present disclosure.

FIG. 25F depicts voltage characteristics vs time for the wireless electrostimulation output of a P3D-eskin system at 20% stretching state according to certain embodiments of the present disclosure.

FIG. 25G depicts voltage characteristics vs time for the wireless electrostimulation output of a P3D-eskin system at 50% stretching state according to certain embodiments of the present disclosure.

FIG. 25H depicts voltage characteristics vs time for the wireless electrostimulation output of a P3D-eskin system at 100% stretching state according to certain embodiments of the present disclosure.

FIG. 26A illustrates battery-free type of P3D-eskins using NFC technology according to certain embodiments of the present disclosure. The system includes the stretchable LM antenna, stretchable printed LM microcircuit integrated with microchips (MCU for NFC, ADC, sensors), and permeable fiber mats as the encapsulation and the substrate.

FIG. 26B depicts digital images of the Near Field Communication (NFC) P3D-eskins before the encapsulation of permeable super-elastic fiber mat.

FIG. 26C depicts digital images of the NFC P3D-eskins after the encapsulation of permeable super-elastic fiber mat.

FIG. 26D depicts digital images of the stretchable NFC antennas using conventional serpentine copper (Cu) coil stretched at 50% strain.

FIG. 26E depicts digital images of the stretchable NFC antennas intrinsically stretchable LM coil stretched at 50% strain.

FIG. 26F depicts FEA of the stress distribution of stretchable antennas under the biaxial stretching (50% strain) using Cu serpentine and intrinsically stretchable LM.

FIG. 26G depicts inductance of serpentine Cu antenna (5 turns) and intrinsically stretchable LM antenna (10 turns) with the same coverage area as a function of frequency at various strains.

FIG. 26H depicts Q factor of the stretchable LM antenna at various strains.

FIG. 26I depicts phase of the stretchable LM antenna at various strains.

FIG. 26J depicts impedance of the stretchable LM antenna at various strains.

FIG. 26K depicts digital images of the skin inflammatory states after covering with P3D-eskin for 30 min exercise.

FIG. 26L depicts digital images of the skin inflammatory states after covering with PDMS-eskin for 30 min exercise.

FIG. 26M depicts thermal imagery of an adult body using forty NFC P3D-eskin arrays, depicting multi-position body temperature in cool/dry environment.

FIG. 26N depicts thermal imagery of an adult body using forty NFC P3D-eskin arrays, depicting multi-position body temperature in warm/humid environments environment.

FIG. 26O depicts continuous temperature monitoring of an adult body during sleeping using P3D-eskin, PDMS-eskin, and gold standard (commercial infrared thermal imager). The error bands in the figure stand for SD, and the scatter values represent mean value.

FIG. 27A depicts digital images showing the stable electrical interface between the ultrastretchable hLM solder and the rigid components for NFC.

FIG. 27B depicts digital images of stretchable P3D-eskin systems under the tensile strain of 250% and 50% respectively.

FIG. 27C depicts digital images of stretchable PDMS-eskin systems under the tensile strain of 250% and 50% respectively.

FIG. 27D depicts inductance of the structured Cu antenna as the function of frequency at various strains. It can be seen at a same frequency, as the strain increases, the inductance increases.

FIG. 27E depicts inductance of the intrinsically stretchable LM antenna as the function of frequency at various strains. It can be seen at a same frequency, as the strain increases, the inductance increases.

FIG. 28 depicts multi-position physiological monitoring of the human body using 40 near-field communication (NFC) P3D-eskin tags. Black dots in the images show the measuring positions of the body temperature. Graphical User Interface (GUI) on a mobile device shows real-time and continuous monitoring using the NFC P3D-eskin tags.

FIG. 29 depicts stress concentration factor from finite element analysis (FEA) as the function of the modulus input of LM circuit.

FIG. 30A depicts impedance of an intrinsically stretchable LM antenna as the function of frequency at various working distances according to certain embodiments of the present disclosure.

FIG. 30B depicts phase of the intrinsically stretchable LM antenna of FIG. 30A as the function of frequency at various working distances.

FIG. 31 depicts continuous monitoring of body temperature during some daily activities. By using NFC P3D-eskin tags, body temperature signals can be stably monitored using a customized mobile application during some daily activities including sitting, walking, and exercising.

FIG. 32A depicts infrared images showing cooling loads and thermal impacts of the P3D-eskin and the PDMS-eskin in a cool environment: (a) onset of thermal monitoring, (b) on-skin attachment of eskins, (c) removal of eskins, and (d) removal of eskins after 1 min.

FIG. 32B depicts digital images showing the on-skin attaching positions of P3D-eskin and PDMS-eskin, and their weights.

FIG. 33A is a schematic illustration of the structure of the ten-layered P3D-eskin. Vertical dashed lines indicate the position of VIAs.

FIG. 33B depicts digital images showing the layer-by-layer fabrication of ten-layered P3D-eskin by in situ electrospinning fiber mat and micropatterning LM circuit repeatedly.

FIG. 33C depicts a digital image showing the overall thickness of the ten-layered P3D-eskin.

FIG. 33D depicts digital images showing all the stretchable layers and VIAs of ten-layered P3D-eskin visualized by the backlight of a panel. Ten layers of LM traces in the shapes of “E, L, E, C, T, R, O, N, I, C” in each layer are connected by nine layers of LM VIAs. 6 LEDs are interconnected by the “E” shaped LM circuit and mounted with the hybrid LM solder on the 1st layer of the P3D-eskin. The anodes of LEDs are connected directly to the positive terminal (5.2 V, by external DC power supply) on the 1st layer. The cathodes are connected to the negative terminal (ground) on the 10th layer through all the nine interlayers and VIAs. The luminance of the 6 LEDs maintains good stability when the sample is stretched to different strains (up to 100%), indicating the reliability of the ten-layered stretchable LM circuit.

FIG. 34 illustrates a permeable bioelectronic system according to certain embodiments of the present disclosure.

FIG. 35 illustrates a permeable bioelectronic system according to certain embodiments of the present disclosure.

FIG. 36 illustrates a method for making a permeable bioelectronic system according to certain embodiments of the present disclosure.

### DETAILED DESCRIPTION

The present disclosure will now be described with reference to the following examples which should be considered in all respects as illustrative and non-restrictive.

Throughout the description and the claims, the words “comprise”, “comprising”, and the like are to be construed in an inclusive sense as opposed to an exclusive or exhaustive sense; that is to say, in the sense of “comprising, but not limited to”.

Furthermore, as used herein and unless otherwise specified, the use of the ordinal adjectives “first”, “second”, etc., to describe a common object, merely indicate that different instances of like objects are being referred to, and are not intended to imply that the objects so described must be in a given sequence, either temporally, spatially, in ranking, or in any other manner.

Example embodiments relate to permeable bioelectronic systems and methods for making the same with one or more technical advantages.

To date, the development of three-dimensional (3D) stretchable electronics is still in its early stage. The present inventors have recognized several critical challenges in this field. First, it is technically challenging to create robust interfaces between rigid components (e.g., inorganic electronic circuit components) and stretchable circuits in the 3D space or stacks. Second, fabrication of the 3D stretchable electronics nowadays is mainly based on the multilayered sequential casting of elastomeric films, such as polydimenthyl siloxane (PDMS), and the bonding of rigid chips. The thickness of the entire 3D stack is typically thicker than 1 millimeter (mm). The conformability and stretchability are significantly deteriorated in comparison to the single-layer electronic skin. Third, the thick and thin-film-based 3D stacks lack permeability, which is unsatisfactory in terms of wearing comfort and chronic biocompatibility. The present inventors have further recognized it is technically challenging to integrate functional electronic components with fiber mats for highly integrated, stretchable, and permeable electronics.

Example embodiments solve one or more of these problems associated with the existing techniques by overcoming one or more of technical obstacles including but not limited to those as mentioned above.

One or more embodiments comprises a permeable bioelectronic system. The permeable bioelectronic system comprises stretchable multilayered circuits comprising liquid metal (LM) and LM interconnects for bonding one or more inorganic electronic circuit components to the stretchable multilayered circuits. In some embodiments, the LM comprise gallium, a gallium alloy, or a mixture thereof. In some further embodiments, the LM comprises eutectic gallium indium alloy (EGaIn), gallium indium tin alloy (GaInSn), or a mixture thereof.

One or more embodiments provide an intrinsically permeable, three-dimensional integrated electronic skin (P3D-eskin), which hybrids high-density inorganic electronic components with organic stretchable fibrous substrates using 3D-patterned, multilayered LM circuits and ultrastretchable hybrid LM solder. The P3D-eskin leverages skin-like softness and durability, fabric-like permeability, and chronic biocompatibility with complex system-level functions including stable sensing, signal processing and analysis, intervention, and wireless communication.

One or more embodiments provide a P3D-eskin system that hybrids inorganic electronic components with organic stretchable fibrous substrates in a 3D architecture that is similar to high-density 3D integrated circuit boards. The P3D-eskin system has stable and complex electronic functions (sensing, signal processing and analysis, intervention, or wireless communication), skin-like softness and stretchability, and outstanding permeability for continuous and comfortable physiological monitoring and interventions.

One or more embodiments provide one or more stretchable integrated electronic systems that realize high density and multi-functions of stretchable electronics and are advantageous over the prior art systems as shown in Table 1 below. From the table it can be seen the permeable bioelectronic system according to certain embodiments has substantially improved stretchability and permeability.

TABLE 1
Comparing system performance between the state-of-the-art integrated flexible and stretchable electronic systems
and the permeable bioelectronic system according to certain embodiments of the present disclosure.

| | Max. | | | | | | |
|---|---|---|---|---|---|---|---|
| | Stretch | Permeability | | | | Bonding | |
| Strategy | ability | (g/m²/day) | Layout | Function | Components | sites | Ref. |
| Wired with | 0% | Impermeable | Multilayer | Wireless data transmission | and | 45~413 | 1, 2 |
| rigid printed | | | | analysis | 21~154 | | |
| circuit boards | | | | | | | |
| (PCBs) | | | | | | | |
| Wired with | Flexible | Impermeable | Single and | Ditto | 24~70 | 104~212 | 3, 4 |
| flexible PCBs | | | multiple | | | | |
| (PI-based) | | | layer | | | | |
| Soft | 100% | Impermeable | Single | Wireless data | 22 | 66 | 5 |
| microfluidic | | | layer | acquisition, | | | |
| assemblies | | | | analysis, and | | | |
| | | | | transmission | | | |
| Serpentine | 16% | Impermeable | Single | Ditto | 31 | 86 | 6 |
| circuits/silicone | | | layer | | | | |
| Serpentine | 30% | Impermeable | Single | Ditto | 18 | 40 | 7 |
| circuits/silicone | | | layer | | | | |
| Helically coiled | 50% | Impermeable | Single | Ditto | 50 | 500 | 8 |
| circuits/silicone | | | layer | | | | |
| 3D integrated | 35% | Impermeable | Multilayer | Ditto | 56 | 200 | 9 |
| electronics | | | | | | | |
| Stacked 3D | 20% | Impermeable | Multilayer | Ditto | 42 | 140 | 10 |
| electronics | | | | | | | |
| Laser-printed | 62.7% | Impermeable | Single | Sensing and | 7 | 28 | 11 |
| LM circuit | | | layer | wearable computing | | | |
| LM NFC | 32% | Impermeable | Single | Wireless motion | 3 | 9 | 12 |
| | | | layer | sensing | | | |
| Pol-gel | 500% | Impermeable | Single | Wireless sensing | 60 | N/A | 13 |
| soldering | | | layer | and communication | | | |
| 3D printed LM | 200% | Impermeable | Single | Sensing with | 7 | 20 | 14, |
| circuit | | | layer | readout circuitry | | | 15 |
| Metal- | 150% | Impermeable | Multilayer | Multifunctional | 15 | 54 | 16 |
| hygroscopic | | | | sensing | | | |
| soldering | | | | | | | |
| Biphasic LM | 400% | Impermeable | Multilayer | Sensing with | 7 | 26 | 17 |
| circuit | | | | amplifying circuitry | | | |
| Solidified LM | 120% | Impermeable | Multilayer | Wireless motion | 22 | 59 | 18 |
| | | | | sensing | | | |
| LMP elastic | 150% | Impermeable | Multilayer | Sensing with | 38 | 130 | 19 |
| PCB | | | | amplifying circuitry | | | |
| P3D-eskin | 550% | 676 | Multilayer | Wireless electrical | 64 | 212 | This |
| | | | | stimulation and | | | work |
| | | | | sensing | | | |

The last column of Table 1 refers to prior art references except the last row, as described below:
1. Jun, J. J. et al. Fully integrated silicon probes for high-density recording of neural activity. Nature 551, 232-236 (2017).
2. Gao, W. et al. Fully integrated wearable sensor arrays for multiplexed in situ perspiration analysis. Nature 529, 509-514 (2016).
3. Niu, S. et al. A wireless body area sensor network based on stretchable passive tags. Nat. Electron. 2, 361-368 (2019).
4. Jiang, Y. et al. A universal interface for plug-and-play assembly of stretchable devices. Nature 614, 456-462 (2023).
5. Xu, S. et al. Soft microfluidic assemblies of sensors, circuits, and radios for the skin. Science 344, 70-74 (2014).
6. Chung, H. U. et al. Binodal, wireless epidermal electronic systems with in-sensor analytics for neonatal intensive care. Science 363, 947 (2019).
7. Kim, J. et al. Battery-free, stretchable optoelectronic systems for wireless optical characterization of the skin. Sci. Adv. 2, e1600418 (2016).
8. Jang, K. I. et al. Self-assembled three dimensional network designs for soft electronics. Nat. Commun. 8, 15894 (2017).
9. Huang, Z. et al. Three-dimensional integrated stretchable electronics. Nat. Electron. 1, 473-480 (2018).
10. Song, H. et al. Systems Based on Stacked Multilayer Network Materials. Sci. Adv. 3785, eabm3785 (2022).
11. Lu, T., Markvicka, E. J., Jin, Y. & Majidi, C. Soft-Matter Printed Circuit Board with UV Laser Micropatterning. ACS Appl. Mater. Interfaces 9, 22055-22062 (2017).
12. Jeong, Y. R. et al. A skin-attachable, stretchable integrated system based on liquid GaInSn for wireless human motion monitoring with multi-site sensing capabilities. NPG Asia Mater. 9, e443 (2017).
13. Lopes, P. A., Santos, B. C., de Almeida, A. T. & Tavakoli, M. Reversible polymer-gel transition for ultra-stretchable chip-integrated circuits through self-soldering and self-coating and self-healing. Nat. Commun. 12, 4666 (2021).
14. Votzke, C., Daalkhaijav, U., Menguc, Y. & Johnston, M. L. 3D-Printed Liquid Metal Interconnects for Stretchable Electronics. IEEE Sens. J. 19, 3832-3840 (2019).
15. Varnava, C. Liquid metals take stretchable circuits to new heights. Nat. Electron. 2, 52 (2019).
16. Tang, L. et al. Metal-hygroscopic polymer conductors that can secrete solders for connections in stretchable devices. Mater. Horizons 7, 1186-1194 (2020).
17. Liu, S., Shah, D. S. & Kramer-Bottiglio, R. Highly stretchable multilayer electronic circuits using biphasic gallium-indium. Nat. Mater. 20, 851-858 (2021).
18. Li, G. et al. Three-dimensional flexible electronics using solidified liquid metal with regulated plasticity. Nat. Electron. 6, 154-163 (2023).
19. Lee, W. et al. Universal assembly of liquid metal particles in polymers enables elastic printed circuit board. Science 378, 637-641 (2022).
   This work: the permeable bioelectronic system according to certain embodiments of the present disclosure.

One or more embodiments provide a permeable, 3D integrated electronic skin (P3D-eskin). The P3D-eskin shapes the impermeable and rigid printed circuit board (PCB) into a skin-like stretchable, soft, and breathable design form factor while maintaining complex system-level functions including multi-position physiological data acquisition, signal processing and analysis, intervention, and wireless communication with a mobile device continuously and comfortably.

According to one or more embodiments, the P3D-eskin comprises a micropatterned permeable and stretchable multilayered circuit board comprising liquid metal (LM) and fiber mats, robust bonding between the functional rigid IC components and the soft LM interconnects using ultrastretchable hybrid LM (hLM) solder for ensuing stable stretchability without electrical failure, and 3D integration among different layers by engineering the vertical penetration of LM to form stretchable vertical interconnect accesses (VIAs).

According to one or more embodiments, comparing with those prior art thin-film-based stretchable 3D electronics, the proposed P3D-eskin-based systems or P3D-eskin platforms enable unprecedented air and moisture permeability, reduce the system-level thickness by ˜54%, improves the softness by ˜60%, and prevent skin inflammation over long-term skin attachment. These systems or platforms also outperform those prior art permeable electronics in terms of an advanced, complex, and monolithic system-level integration that avoids the use of external PCBs (Table 2).

TABLE 2
Comparing system performance between the state-of-the-art permeable integrated electronics and
the permeable bioelectronic system according to certain embodiments of the present disclosure.

| | | | | | System- | | Integration | |
|---|---|---|---|---|---|---|---|---|
| | Max. | Permeability | | | on-a- | Wireless | density | |
| Strategy′ | strain | (g/m²/day) | Layout | Function | chip | communication | (/cm²) | Ref. |
| Discretely | 30%~80% | N.A. | Single | Multi- | Yes | No | 0.02~1 | 20-22 |
| integrated | | | layer | functional | | | | |
| e-textile | | | | sensing | | | | |
| Textile | 40% | N.A. | Multilayer | Wireless | No | Only | ~0.035 | 23 |
| PCB | | | | multi- | | transmission | | |
| | | | | functional | | | | |
| | | | | sensing | | | | |
| Porous | 30% | N.A. | | | No | No | 4 | 24 |
| membrane | | | | | | | | |
| based | | | Single | LED | | | | |
| circuit | | | layer | circuit | | | | |
| Chip-less | 10% | 676 | Single | Wireless | No | No | 1.99 | 25 |
| | | | layer | sensing | | | | |
| wireless | 550% | N.A. | | Wireless | | Yes | 5.33 | This |
| eskin | | | | electrical | | | | work |
| P3D-eskin | | | Multilayer | stimulation | Yes | | | |
| | | | | and | | | | |
| | | | | sensing | | | | |

The last column of Table 2 refers to prior art references except the last row, as described below:
20. Wicaksono, I. et al. A tailored, electronic textile conformable suit for large-scale spatiotemporal physiological sensing in vivo. npj Flex. Electron. 4, 5 (2020).
21. Choi, H. W. et al. Smart textile lighting/display system with multifunctional fibre devices for large scale smart home and IoT applications. Nat. Commun. 13, 814 (2022).
22. Lin, R. et al. Digitally-embroidered liquid metal electronic textiles for wearable wireless systems. Nat. Commun. 13, 2190 (2022).
23. Yang, Y. et al. A non-printed integrated-circuit textile for wireless theranostics. Nat. Commun. 12, 4876 (2021).
24. Jeong, G. S. et al. Solderable and electroplatable flexible electronic circuit on a porous stretchable elastomer. Nat. Commun. 3, 977 (2012).
25. Kim, Y. et al. Chip-less wireless electronic skins by remote epitaxial freestanding compound semiconductors. Science 377, 859-864 (2022).
   This work: the permeable bioelectronic system according to certain embodiments of the present disclosure.

According to one or more embodiments, it has been demonstrated that the fabrication of a series of functional permeable bioelectronics using the P3D-eskin platform to continuously record and wirelessly transmit multi-position physiological signals of human bodies.

One or more embodiments achieve one or more technical advantages over prior art system. Those technical advantages may be one or more of the following, including but not limited to, more comfortable and biocompatible for long-term wearing/implanting bioelectronics, more complex system-level functions, more accurate and reliable in the warble signal acquisition, more durable under various wearable/implanting deformations, etc.

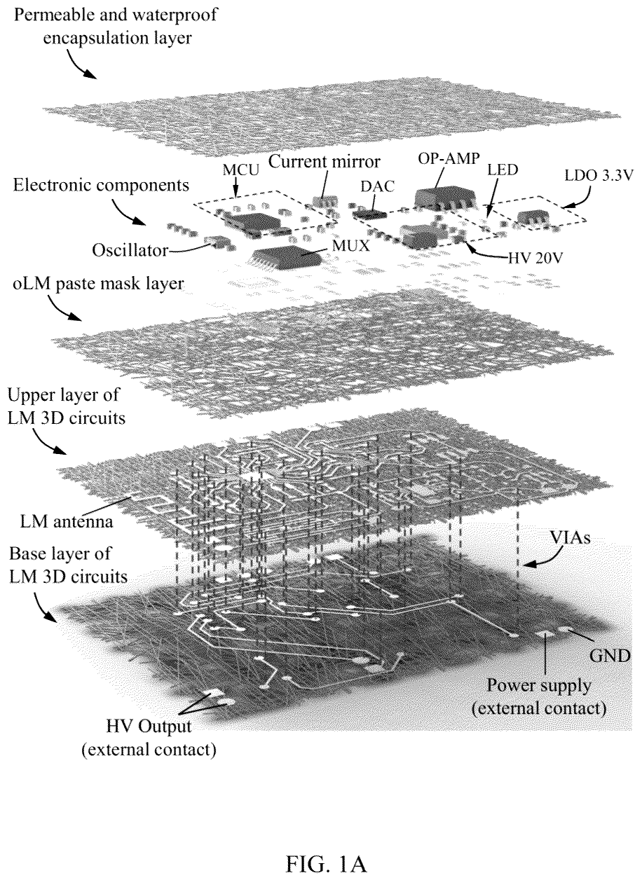

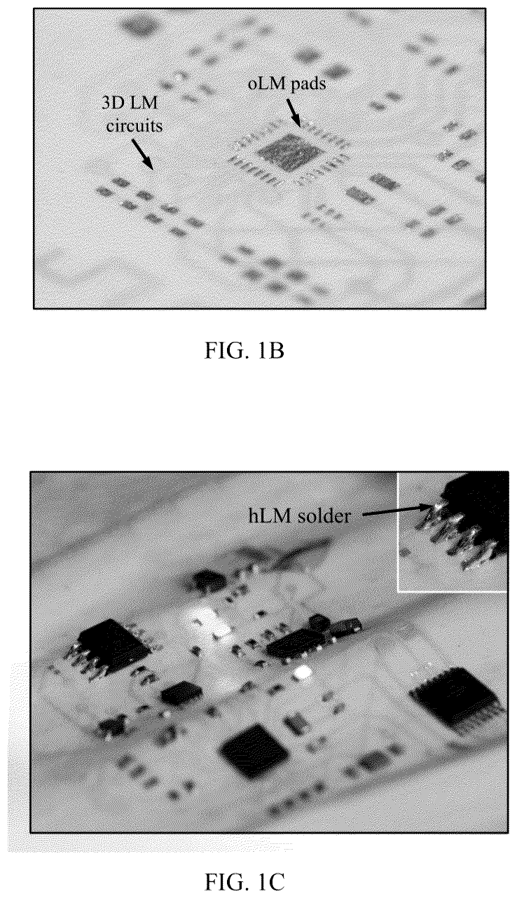

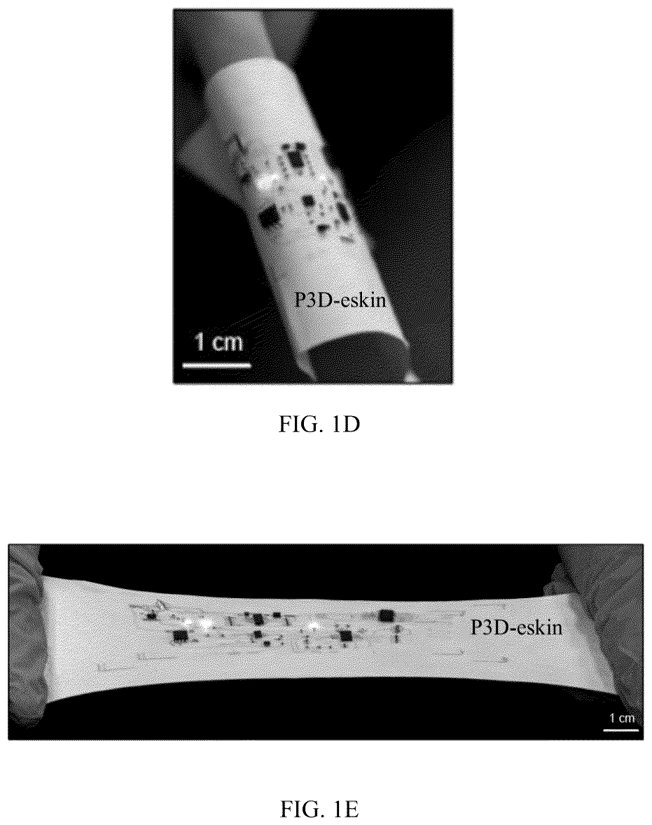

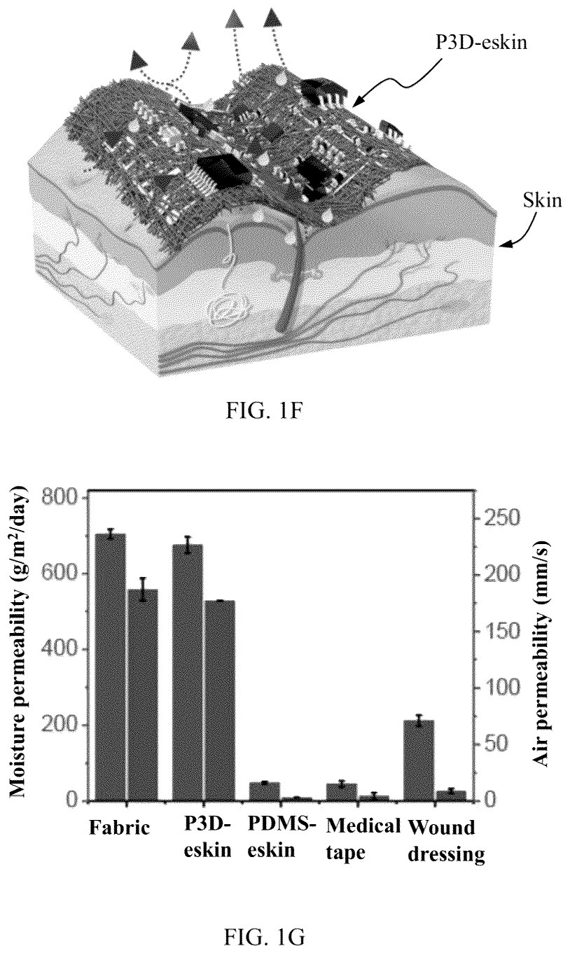

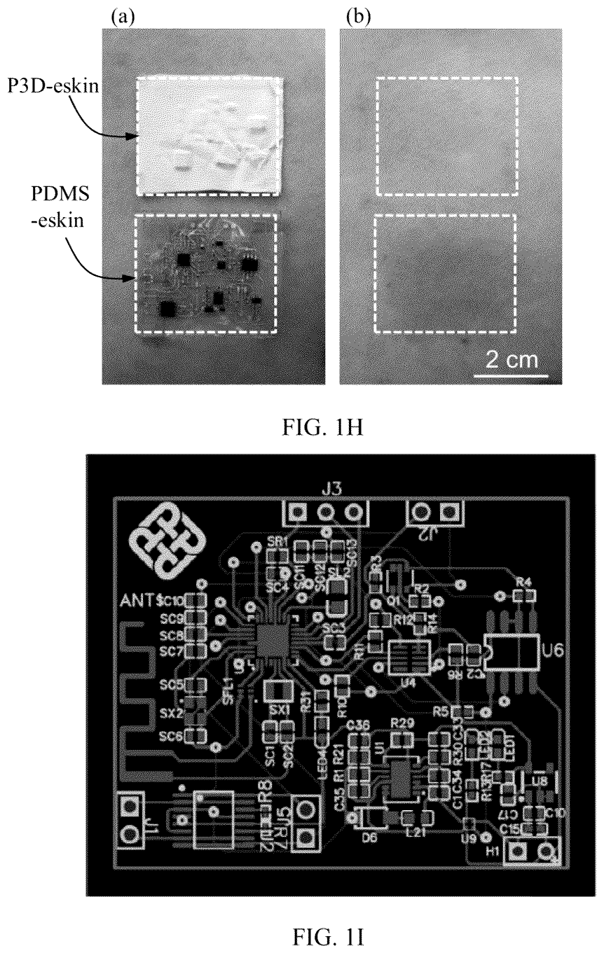

Referring to FIG. 1A and FIG. 1I, by way of example and for illustrative purpose only, the P3D-eskin comprises four stretchable and permeable layers. As illustrated, the multilayered P3D-eskin comprises a base circuit layer (which may also be called base layer in some embodiments) in the form of a base LM circuit layer, an upper circuit layer (which may also be called upper layer in some embodiments) in the form of an upper LM circuit layer, a paste mask layer, and an encapsulation layer. In the present embodiments, the paste mask layer is bonded with rigid electronic components using stretchable hLM solder. The LM may comprise eutectic gallium-based alloy or alloys due to their unlimited stretchability and low modulus as liquid, high electrical conductivity, excellent biocompatibility, and patternability.

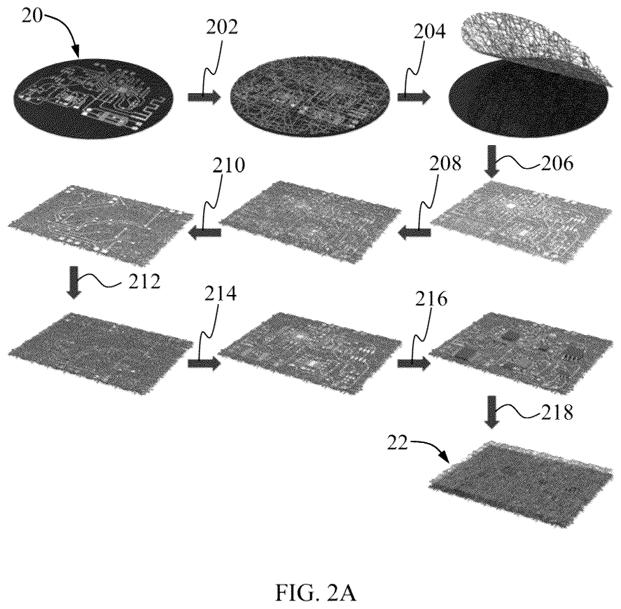

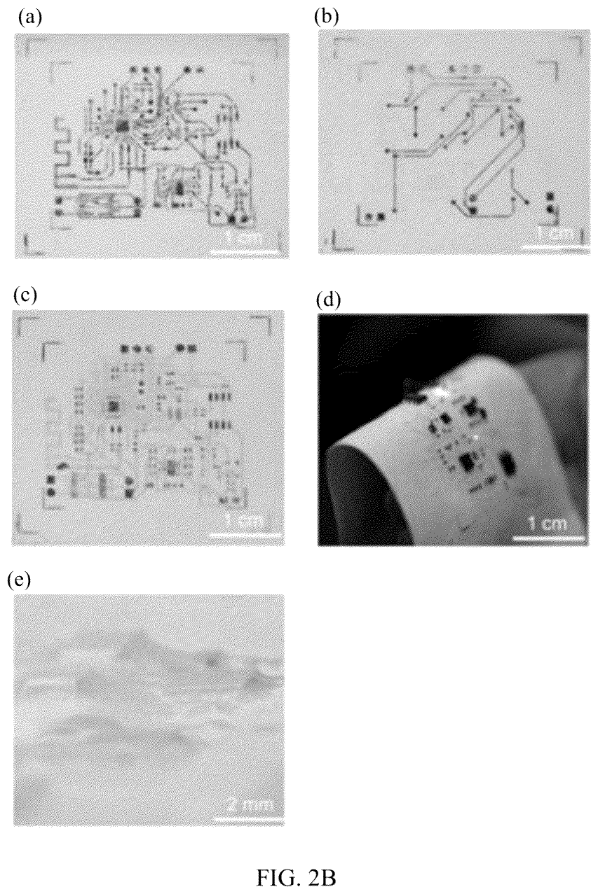

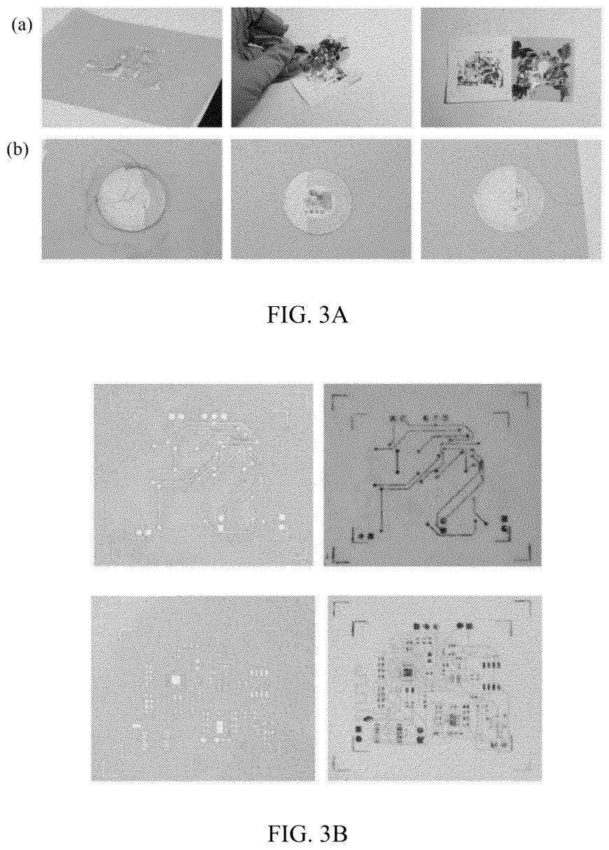

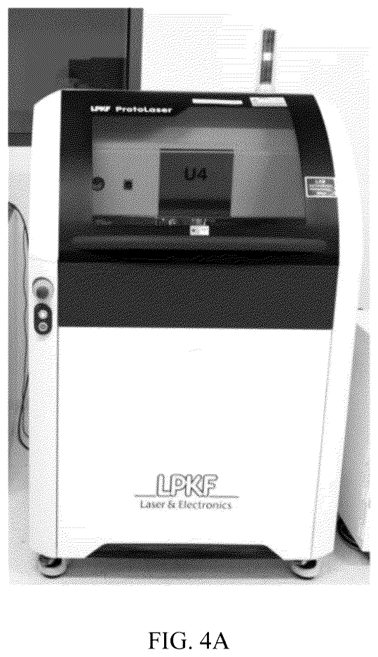

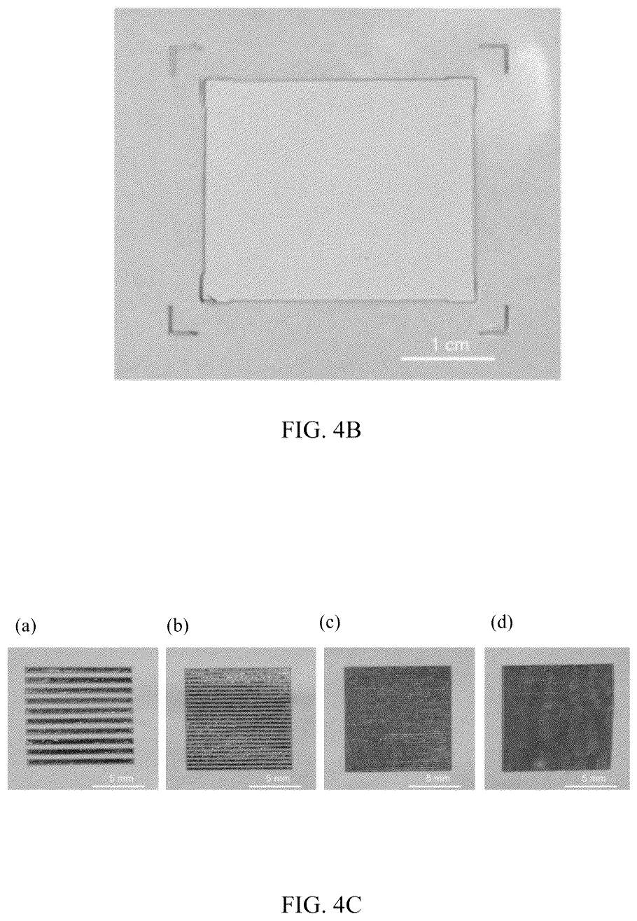

The P3D-eskin may be fabricated in proper processes. An example fabrication procedure is illustrated in FIG. 2A and FIG. 2B. According to one or more embodiments, the base circuit layer (˜100 μm) and the upper circuit layer (˜100 μm) are formed on a stretchable fibrous mat using a combination of photolithography, pattern transfer, and stencil printing process (FIGS. 3A, 3B, 4A and 4B). The thickness of base circuit layer or the upper circuit layer may be different from 100 μm. In some embodiments, the thickness of base circuit layer or the upper circuit layer may be in a range from around 25 μm to around 500 μm, such as in a range from 25 μm to 100 μm, or from 50 μm to 200 μm, or from 100 μm to 400 μm, or from 300 μm to 500 μm, or any other subset of the range from 25 μm to 500 μm.

The base and upper circuit layers comprise LM micropatterns or micropatterned LM. The micropatterned LM serves as electrical means, such as stretchable antenna, interconnects, pads, and/or contacts. The vertical electrical connections between the base and the upper circuit layers are achieved using LM VIAs. Subsequently, rigid electronic components (e.g., inorganic electronic circuit components, such as inorganic semiconductor components or elements) are bonded onto the LM circuits using hLM. The hLM comprises a combination of partially oxidized LM (oLM) and LM. The OLM is formed (e.g. printed) on the paste mask layer made of thin fibrous styrene-butadiene-styrene SBS (˜30 μm), where the thin SBS has been previously formed (e.g. deposited) on the upper circuit layer (FIG. 1B). The thickness of paste mask layer may be different form 30 μm. In some embodiments, the thickness of paste mask layer may be in a range from 10 μm to 50 μm, such as in a range from 10 μm to 30 μm, or from 20 μm to 40 μm, or from 30 μm to 50 μm, or any other subset of the range from 10 μm to 50 μm. In certain embodiments, the paste mask layer may comprise one or more biocompatible elastomeric fibers other than the fibrous SBS. The one or more biocompatible elastomeric fibers may comprise styrene-isoprene-styrene block copolymer, a styrene-polybutadiene-styrene block copolymer, a styrene-butadiene block copolymer, a poly(styrene-block-butadiene-block-styrene) copolymer, a polyisoprene rubber, a butadiene rubber, a polyurethane, a thermoplastic polyurethane, a polyvinyl alcohol, a polycaprolactone, polycaprolactone, or a mixture thereof.

The rigid electronic components may comprise, including but not limited to, one or more of light-emitting diodes (LEDs), microcontroller unit (MCU), oscillator, multiplexer (MUX), current mirror, digital-analog-convertor (DAC), operational amplifier (OP-AMP), high voltage module (HV, 20 V), and low dropout regulator (LDO, 3.3 V). The rigid electronic components may be adhered onto the printed oLM pads. Additional LM pastes may be applied on the pin/oLM interfaces (FIG. 1C). The encapsulation layer (˜50 μm) may be directly electrospun to cover the entire 3D hybrid electronic circuit conformally. The thickness of encapsulation layer be different from 50 μm. In some embodiments, the thickness of encapsulation layer may be in a range from 50 μm to 500 μm, such as in a range from 50 μm to 100 μm, or from 100 μm to 300 μm, or from 200 μm to 400 μm, or from 300 μm to 500 μm, or any other subset of the range from 50 μm to 500 μm. The encapsulation layer may comprise permeable but waterproof SBS mat.

According to one or more embodiments, for purpose of fabricating the P3D-eskin, processing solvents are used as received. Dextran (Sigma-Aldrich), LM, implemented as eutectic GaIn, (LM, melting point 15.7° C., Sigma-Aldrich), negative photoresist (NR9-1500P, Futurrex, Inc., USA), developer for NR9-1500P (DR6, Futurrex, Inc., USA), poly(styrene-block-butadiene-block-styrene) (SBS, Kraton) are used as received.

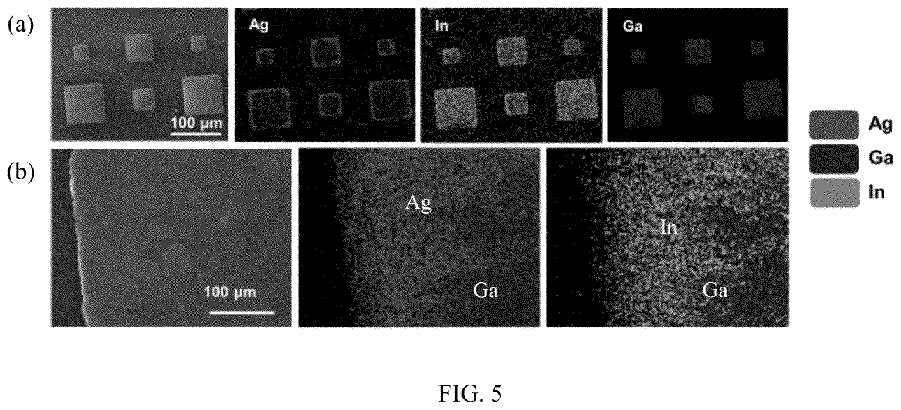

By way of example, the fabrication procedure of multilayered LM circuits combines the photopatterning-pattern transfer-selective wetting method and the stencil printing of LM. As such, the fabrication takes advantages of both photopatterning and stencil printing techniques. (FIGS. 3A, 3B, 4A, 4B and 4C). Permeable and stretchable LM microelectrodes are patterned and function as stretchable antennas, traces, connections, and/or contacts for the microcircuits. Referring to FIG. 2A, by way of example, a sacrificial layer is prepared on the wafer by spin coating of a dextran solution (10 wt % in water) at 4000 revolutions per minute (rpm) for 40 s. After baking treatment at 80° C. for 1 min and then 180° C. for 30 min, a negative photoresist (NR9-1500P) is subsequently spin-coated on the dextran-coated wafer, followed by the photolithography and developing process. Silver (Ag) microcircuit 20 (acting as the upper layer of the 3D circuit) is generated using the lift-off treatment of a deposited Ag film (300 nm thick) by thermal evaporation. At step 202, a fibrous SBS mat (100 μm thick, insulating layer) is directly electrospun on the Ag microcircuit 20. The polymer solution is prepared by dissolving the SBS polymer with a weight ratio of 13 wt % in the mixed solvent (tetrahydrofuran/dimethylformamide=3:1). The voltage is set as 18 kV and the collecting distance is 15 cm. After dissolving the dextran layer with deionized water, at step 204, Ag microcircuit 20 is then transferred to the SBS mat. At step 206, the Ag microcircuit layer is selectively wetted with LM in a glovebox, cut into square-shaped pieces, and at step 208, covered with a thin electrospun SBS mat (˜30 μm thick, paste mask layer). The selective wetting of LM lies in contrast between the LM-lyophobic property of the SBS mat and the LM-lyophilic property of Ag. In the fabrication of the LM microcircuit, EGaIn (eutectic GaIn) wets only the Ag-covered areas because of reactive alloying, and dewets from the SBS surface because of the high intrinsic surface tension of LM. When applying EGaIn on Ag, reactive alloying between Ag and Indium (In) forms AgIn alloys. Additional EGaIn will subsequently wet the AgIn alloy layer and form the EGaIn/AgIn/Ag trilayer (FIG. 5).

At step 210, this upper circuit layer is then flipped over and LM traces of the base circuit layer is stencil-printed. After electrospinning another SBS mat (100 μm thick) as the substrate, at step 212, the vertical interconnect accesses between two layers of the 3D LM circuits are engineered by laser cutting method (LPKF ProtoLaser U4) and these VIAs are filled with LM. At step 214, the circuit board is flipped over again and the partially oxidized LM (oLM) ink is stencil-printed. The oLM ink is prepared by heating pristine LM in the air at a set temperature of 80° C. for 16 h. The oLM is printed onto the paste mask layer via a customized mask, serving as contact pads for the electronic components. After placing the components on the paste mask layer at step 216, additional pristine LM paste is applied at the pin/oLM interfaces to form the ultrastretchable hybrid LM (hLM) solders. In the present embodiment, the weight ratio between the oLM pad and LM paste is 1:2. In some other embodiments, the weight ratio may be in a range from 1:0.5 to 1:8, such as in a range from 1:0.5 to 1:3, or in a range from 1:2 to 1:6, or in a range from 1:3 to 1:7, or any subset of the range from 1:0.5 to 1:8.

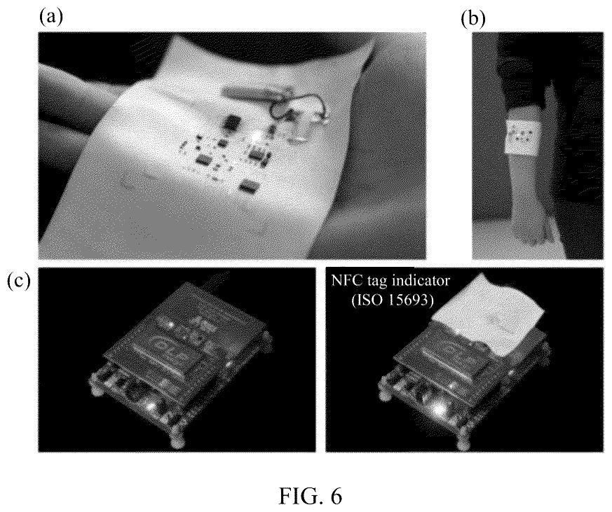

A detailed circuit diagram design and the printed circuit board (PCB) design is illustrated in FIG. 1I. Circuit components in each layer may comprise one or more of microcontroller unit (MCU), oscillator, multiplexer (MUX), current mirror, digital-analog-convertor (DAC), operational amplifier (OP-AMP), high voltage module (HV, 20V), and low dropout regulator (LDO, 3.3 V). For the wireless communication, the P3D-eskin system may be equipped with a Bluetooth (BLE) 5.1 built-in MCU (CC2640, Texas Instruments) and matched 2.4 GHz LM BLE antenna (planar inverted F-shaped Antenna) to achieve data acquisition, transmission, and functional control by simply using a smartphone with a mobile app. Code composer studio (CCS) is used for MCU programming. The Android application used for communication by mobile devices is developed by Android Studio. The power of P3D-eskin is supplied by a lithium-ion battery, and the voltage is regulated by the LDO (FIG. 6). Finally, at step 218, the whole permeable stretchable circuit board is conformally encapsulated with a permeable and waterproof SBS mat to ensure stable operations, thereby completing the P3D-eskin 22.

According to one or more embodiments, for P3D-eskin equipped with Bluetooth functionality, the upper layer of the LM 3D circuit comprises high-density complex LM micropatterns, such as the routing of tracks, pins forming an island or peninsula-shaped path (e.g., a circle), and many densely aligned long tracks. For the battery-free type of P3D-eskin, the stretchable antenna coil is designed compactly with a large turn number. Patterning such complex and high-density micropatterns on permeable, supersoft, and stretchable substrates while maintaining outstanding mechanical, electrical, and electromagnetic performance is extremely challenging because of the large surface roughness and porosity. As such, a combination of method comprising photopatterning, pattern transfer, and selective wetting method is adopted to fabricate the complex and high-density upper circuit layer.

It is challenging for the simple stencil printing technique to create such complex patterns as described herein (FIG. 3A), and the process of stencil printing is very likely to make the mask detached and thus ruins the pattern. Nevertheless, simple patterns such as circuit traces and contacts in the base circuit layer and pads in the paste mask layer (FIG. 3B) can be obtained by a stencil printing technique, which is more cost-effective and time-saving.

According to one or more embodiments, to obtain a relatively high-resolution microscale LM circuit without agglomeration by stencil printing, the present inventors have tackled several technical challenges. Firstly, a high-precision PCB-fabrication-compatible laser cutting machine (LPKF U4, FIG. 4A) with a vacuum table is adopted to fabricate the stencil. In principle, the laser beam focus can reach around 15 μm and thereby provides a powerful tool to fabricate high-resolution stencils. Secondly, the polyimide (PI) thin film (12.5˜25 μm thick) is used as the stencil, so that the stencil is conformally laminated onto the substrates with a fixing frame and markers (FIG. 4B). The densely packed line arrays denote that the smallest trace size from the stencil printing technique is ˜100 μm (50 μm lines displayed numerous disconnections) and the highest trace density of ˜70 lines/cm² (FIG. 4C). This resolution is comparable to the patternability of conventional PCB fabrications of which the typical linewidth is around 10 mil (254 μm). Therefore, the proposed fabrication procedure here is the optimum solution for such complex 3D integrated circuit boards with stretchability, softness, moisture-permeability, and waterproofness.

Fabrication of polydimethylsiloxane eskins (PDMS-eskins). For purpose of reference and comparison to demonstrate the improved performance of the P3D-eskin or P3D-eskins as described herein, 3D eskins are fabricated to have the same device design and configuration using thin PDMS as the substrate, interlayer, and encapsulation material. Firstly, a layer of PDMS (Sylgard®184, 10:1) is spin-coated (500 rpm, 30 s) onto a clean and dry glass sheet, and cured in an oven (80° C., 30 min). Meanwhile, two copper/polyimide (Cu/PI) films (18/12.5 μm) are laser-cut (LPKF ProtoLaser U4) into patterns of the top layer and bottom layers of the circuit respectively. Picked up by water-soluble tapes, their PI side is deposited with Ti/SiO₂ layers (5/100 nm) by electron beam evaporation as the adhesive interface between circuits and the PDMS substrate. After treating both surfaces with ultraviolet ozone (UVO) for 5 min, the base layer of the circuit pattern is transferred onto the PDMS substrate with strong bonding, and then rinsed in water to remove the water-soluble tape. Another layer of PDMS is spin-coated in the same way on top of the bottom layer circuit and cured, which functions as the intermediate insulating layer. The laser cutting method is used to fabricate VIAs on the PDMS layer. The top layer copper circuit is then transferred and printed onto the insulating layer in the same way after Ultraviolet-Ozone (UVO) treatment and aligning with the base layer. Then the VIAs are filled with commercial soldering paste, and electronic components are placed on paste-applied pads. The components are soldered onto the multilayered circuit with a hot wind blower. Finally, the circuit board is fully encapsulated by casting the PDMS solution and curing it in the oven (80° C., 15 min).

Characterizations. The morphology of the LM 3D circuits and surface oxidation states of the oLM is explored using scanning electron microscopy (SEM, TESCAN VEGA3), and X-ray photoelectron spectroscopy (XPS, Thermo Fisher Scientific Xesa) respectively. Both air permeability and moisture permeability tests are performed at constant temperature (22° C.) and humidity (63%). The air permeability tests are conducted according to ASTM D737-08 standard using a MO21S air permeability tester (SDL Americ, Inc.) with an airflow pressure of 100 kPa. Moisture permeability tests are performed according to the standard E96/E96M-13 by the cup method. The testing duration is 72 h. The waterproofness of the P3D-eskin system is characterized by a standard rain test according to AATCC Test Method 35-2006. The sample size is set as 20 cm×20 cm, and the water spraying duration is 2 min. The sweat-resistance tests of the P3D-eskin system are performed by immersing the P3D-eskin system in water and artificial sweat (ZW-HY-1000, pH value: 4.7±0.1, Zhongwei Equipment Co., LTD) with the stirring rate of 300 rpm. The luminance stability of the embedded LEDs inside the P3D-eskin system indicates the sweat resistance of the system. The statistic values (mean, standard deviation (SD)) are obtained with at least three parallel samples. Each sample is tested for at least three times. The mechanical properties of the materials are characterized using a universal testing machine (Instron 5566). The electrical resistance of resistors connected with the LM, oLM, and hLM under different strains is measured by a four-terminal method with a source meter (Keithley 2400) coupled with a customized stretching machine (Zolix). The output and transfer characteristics of the stretchable metal-oxide-semiconductor field-effect transistors (MOSFETs), and multilayer stretchable switch array are characterized using a semiconductor analyzer (Keithley 4200A-SCS Parameter Analyzer) connected with a probe station (Micromanipulator) and a customized stretching setup. The stretchable logic circuits are characterized using a digital oscilloscope (Rigol).

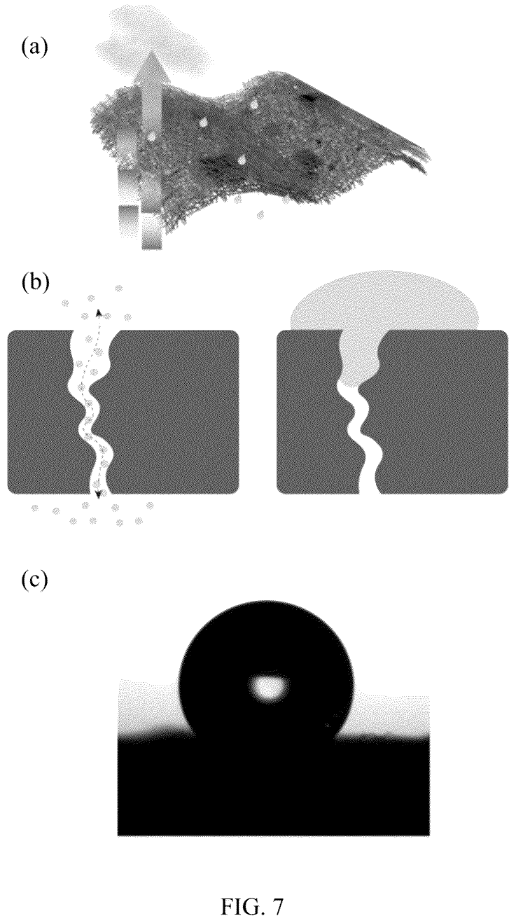

The microporous fibrous structure of the electrospun fiber mat allows air and moisture (e.g., water vapor) to pass through it (FIG. 7A), while the intrinsic hydrophobicity of the SBS fiber mat (FIG. 7B), which shows a large water contact angle (CA) of 127° (FIG. 7C), can repel water droplets. That is, both permeability and waterproofness are achieved.

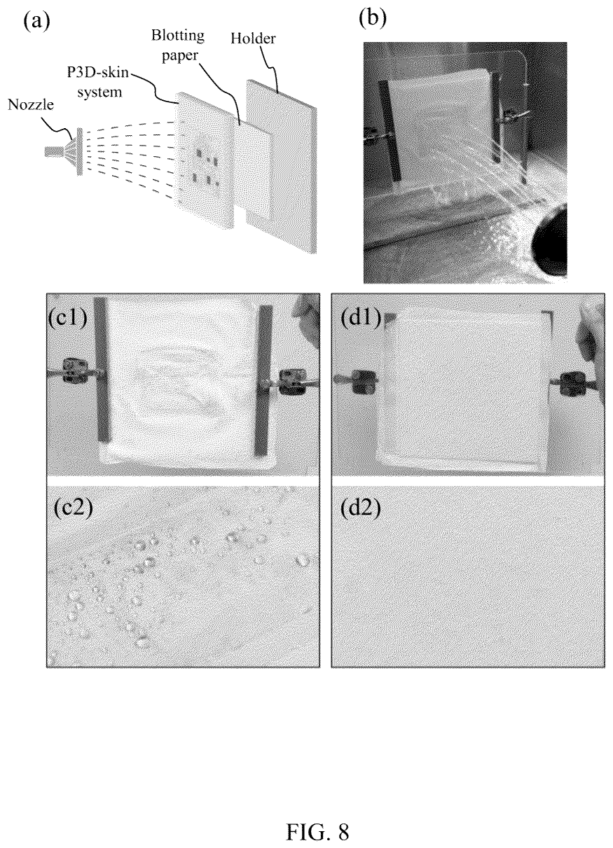

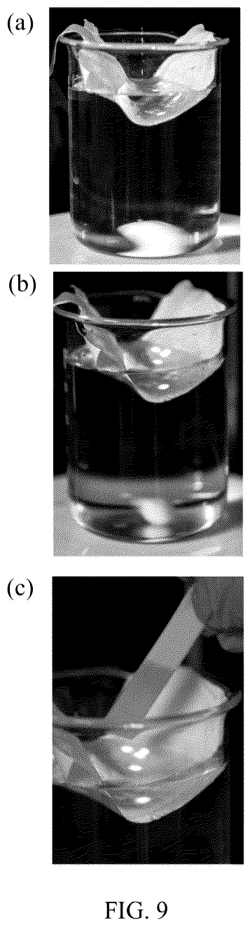

The P3D-eskin is extremely soft (FIG. 1D) and highly stretchable and demonstrates a stable electrical function under a large tensile strain of 550% (FIG. 1E). It offers wireless, continuous, and comfortable physiological monitoring and intervention of the human body through a mobile device interface. Importantly, because the P3D-eksin is fabricated based on the porous and fibrous substrate, interlayer, and encapsulation, it also offers unprecedented permeability (FIG. 1F), in comparison to those impermeable 3D stretchable electronics made with elastic thin films and bulks. The air and moisture permeabilities of P3D-eskin reaches 177 mm/s and 676 g/m²/day, which are 15 folds and 44 folds higher than medical tapes, and 3 folds and 22 folds larger than commonly used wound dressing, respectively (FIG. 1G). The waterproofness of the P3D-eskin system reaches the “Excellent” grade according to the standard rain test. After spraying water onto the front side of the P3D-eskin system for 2 min, no observable water is found on the blotting paper (FIG. 8). Further, the stability of the P3D-eskin system is also tested in water and artificial sweat (pH: 4.7±0.1). An LED-embedded P3D-eskin system is fabricated and immersed in both liquids. Outstanding electrical stability is indicated by the stable luminance of the LEDs both in water and in artificial sweat (FIG. 9)

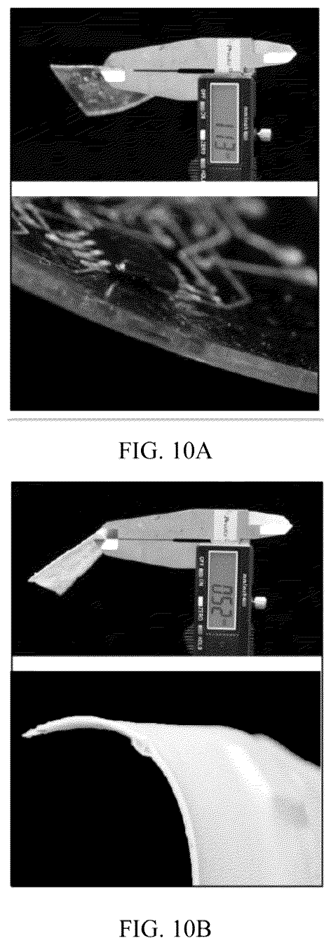

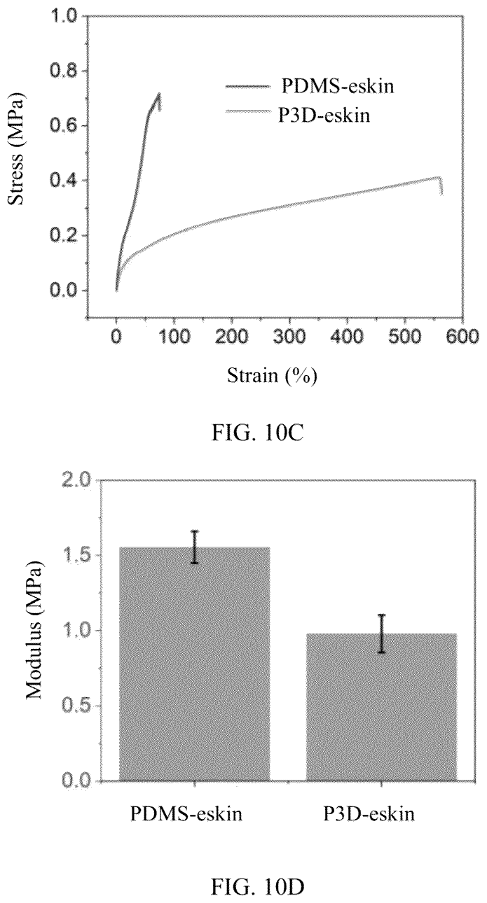

According to one or more embodiments, the P3D-eskin shows outstanding chronic biocompatibility and the skin area covered by the P3D-eskin maintains inflammation-free during one week of on-skin attachment. As a reference, a similar 3D eskin is fabricated that uses thin PDMS as the substrate, interlayer, and encapsulation material. The PDMS-eskin is thicker by ˜54% and more rigid by ˜60%, and shows hardly any permeability to air and a poor moisture permeability below 50 g/m²/day due to the compact and thin-film type of layout following the conventional spin-coating and casting process (FIGS. 10A, 10B, 10C and 10D). Although PDMS is known as a biocompatible material, the impermeability of multilayered PDMS-eskin results in serious skin erythema under the on-skin attachment test (FIG. 1H).

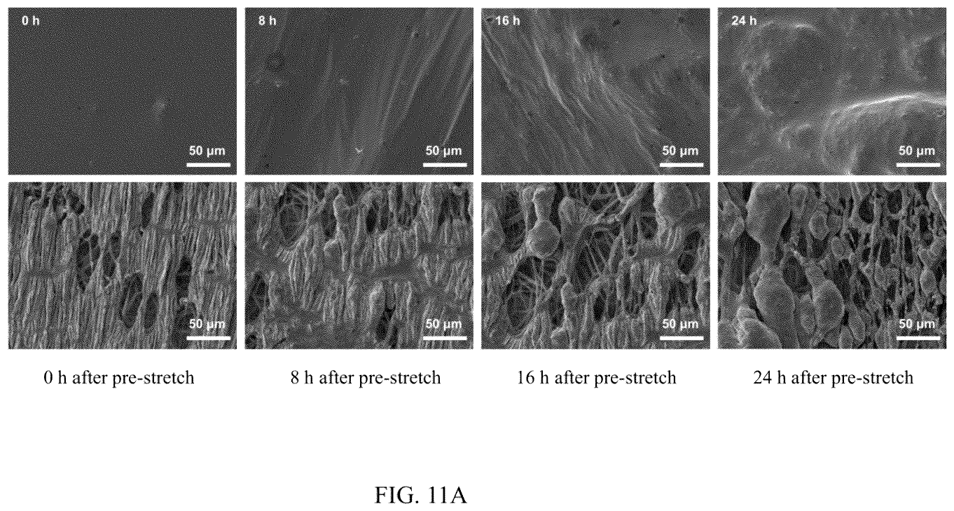

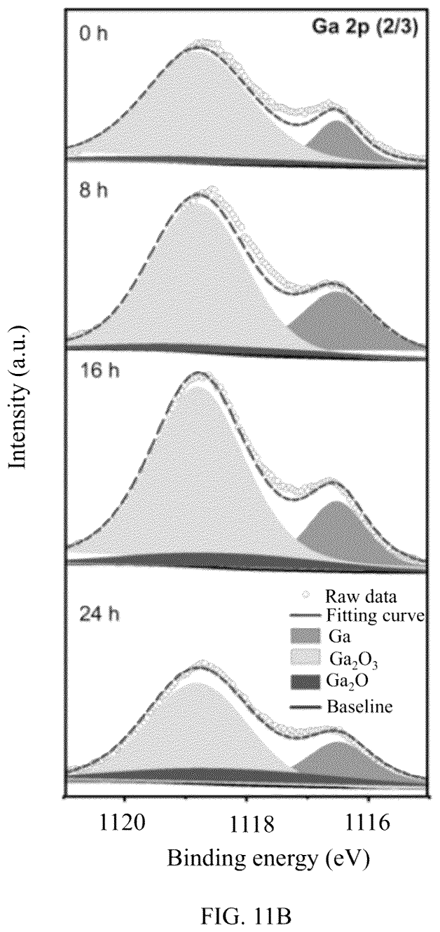

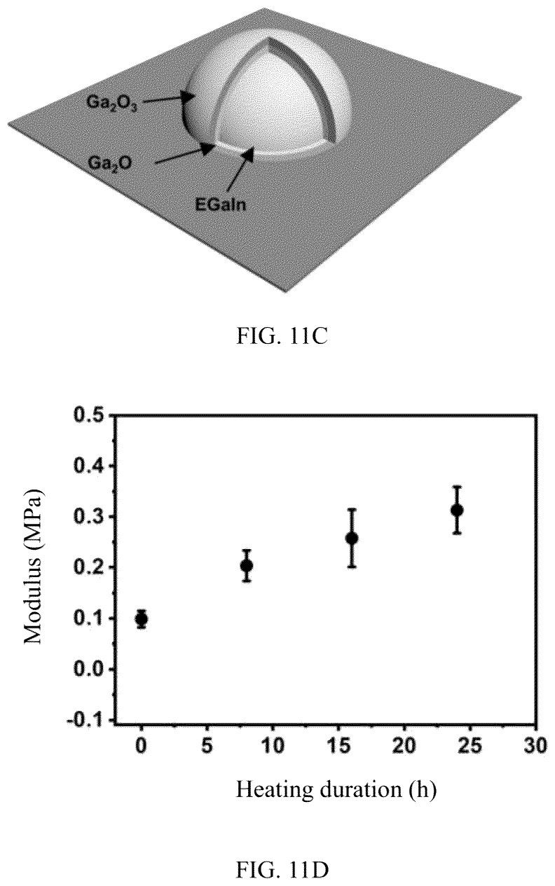

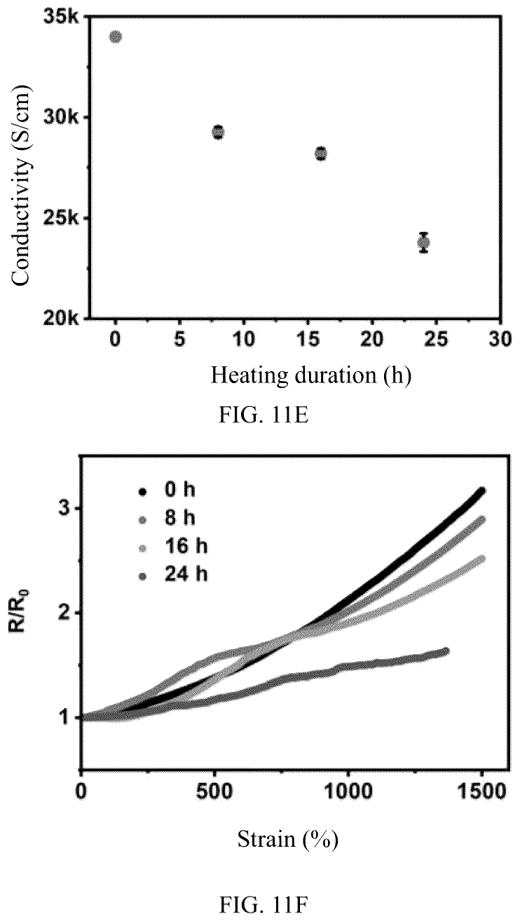

Ultrastretchable hLM solder for reliable 3D interfaces between rigid components and LM circuits. To achieve a high stretchability and stability of the P3D-eskin, the present inventors have recognized that it is of critical challenge to ensure a seamless interface among the different vertical layers that provide necessary electrical insulation and connection, and stable interfaces between the soft LM and the rigid electronic chips that can endure large deformations. To address this challenge, two different kinds of LM inks are formulated, being the pristine LM and oLM (FIG. 11A to FIG. 11F), serving as an ultrastretchable hLM solder for the 3D circuits.

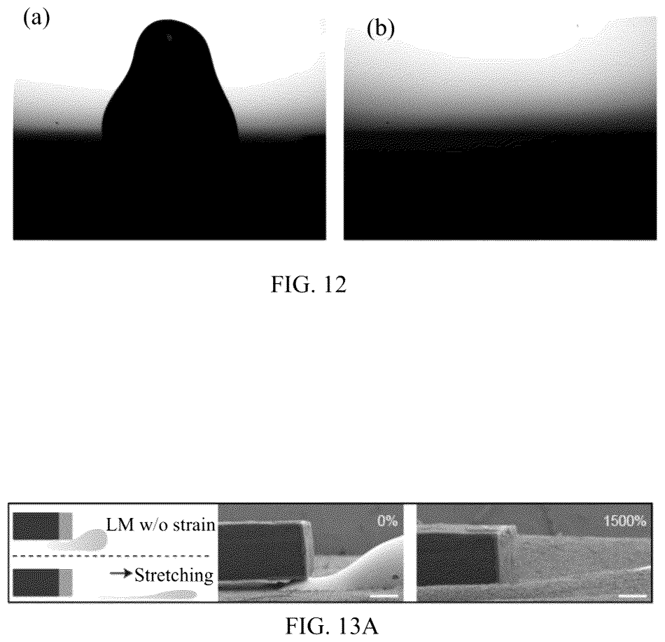

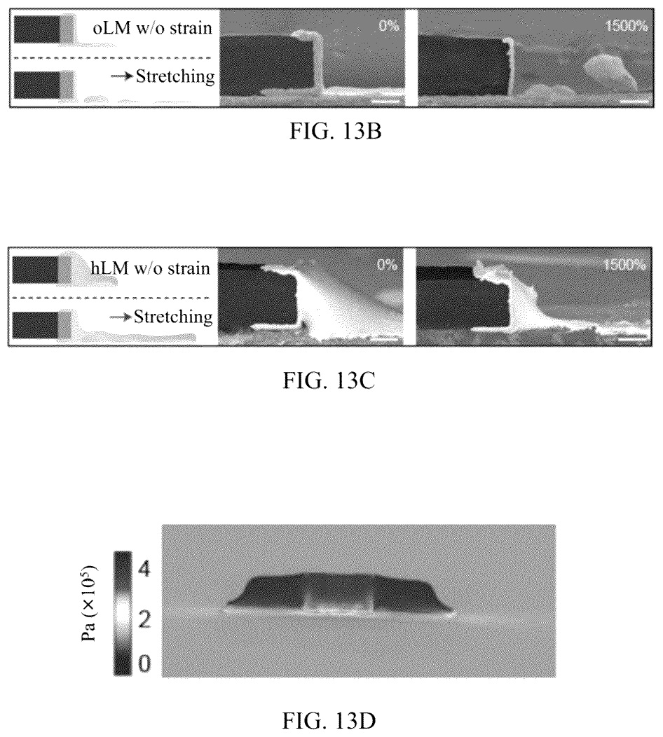

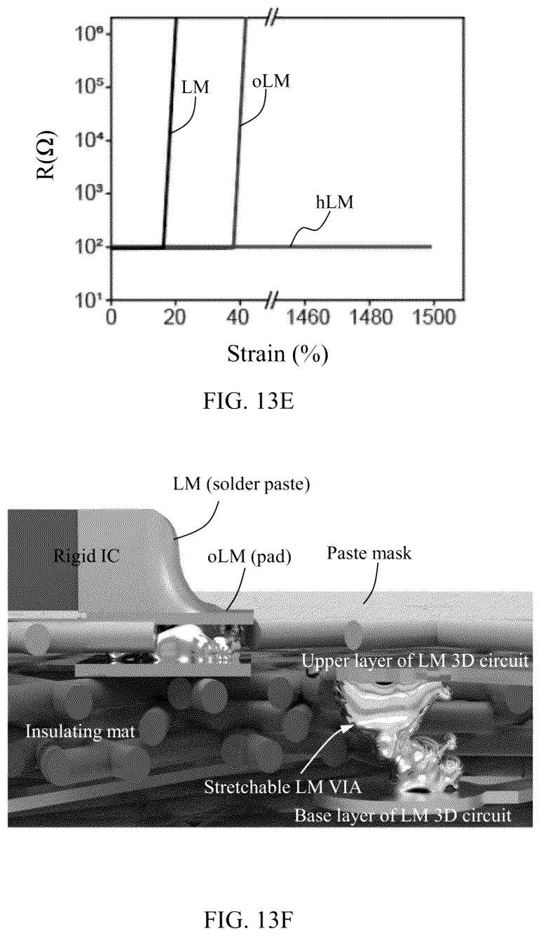

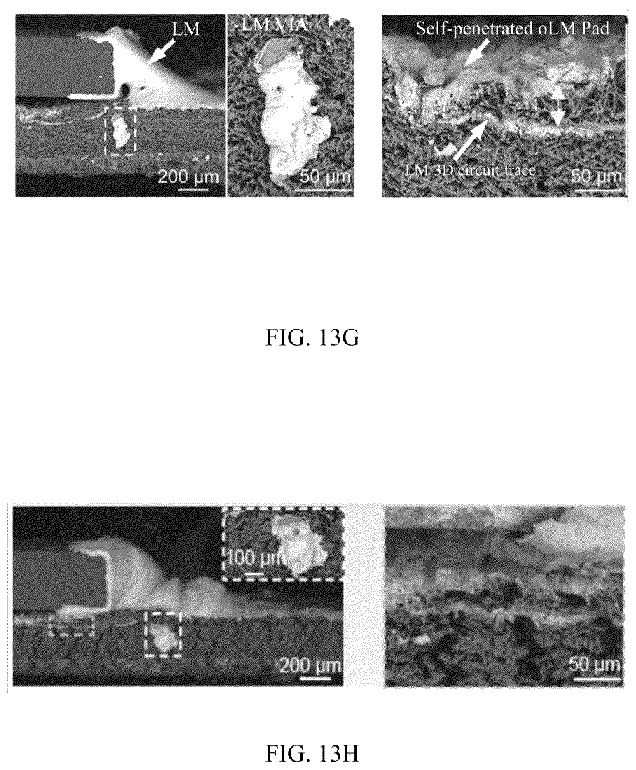

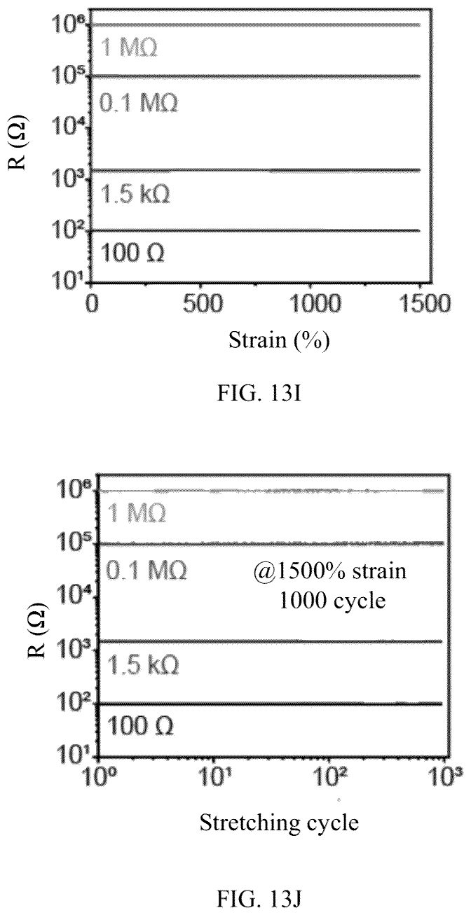

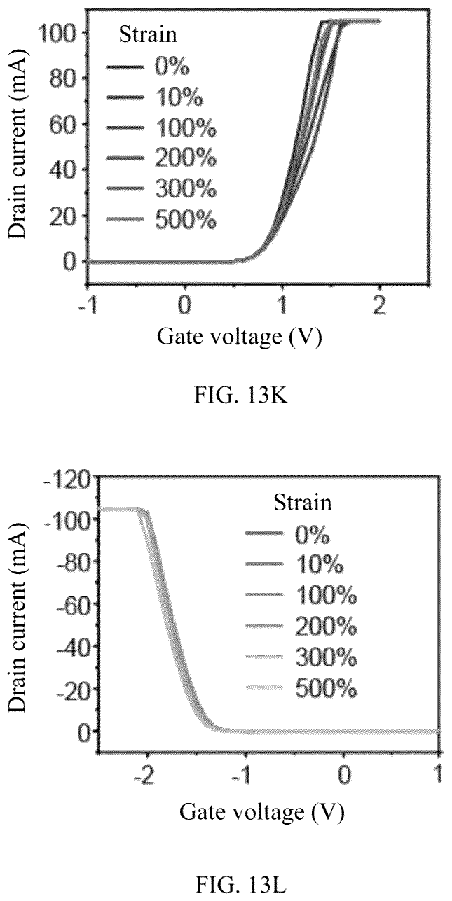

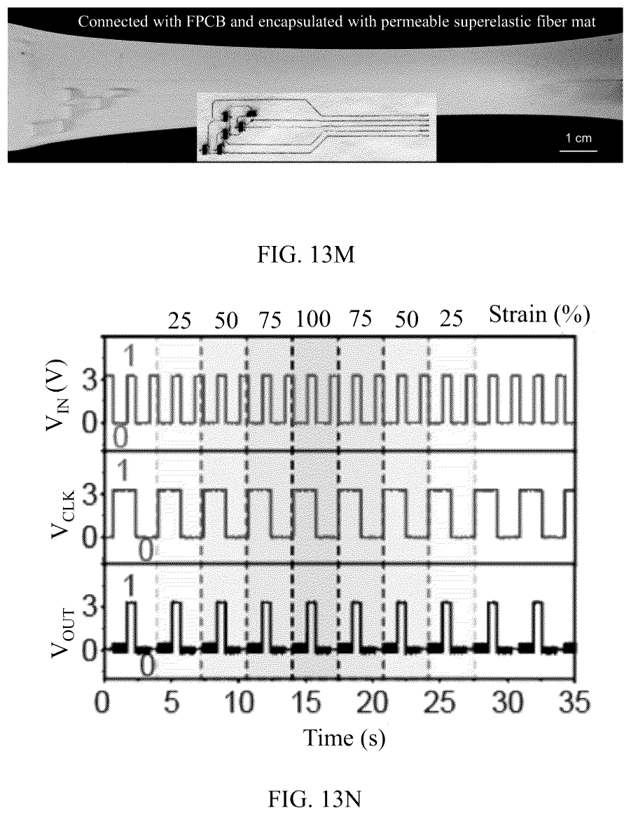

The pristine LM shows high fluidity but low wettability to the fibrous SBS substrate (FIG. 12) and it is used for the fabrication of stretchable circuit antennas, interconnects, and VIAs on the base and upper circuit layers. As such, the base and upper circuits remain outstanding in-plane stretchability and out-of-plane insulation, unless they are connected with VIAs. However, connecting the rigid pins with the pristine LM results in poor stretchability, the pin/LM interface falls apart during stretching due to the dewetting of LM (FIG. 13A). In contrast, the wettability of oLM is much higher because the oxidation has reduced the surface tension of LM. oLM is therefore chosen to print on the paste mask layer as contact pads, providing good adhesion between the underneath soft LM circuits and the rigid pins of the electronic components. Nevertheless, due to the low stretchability of oLM, the pin/oLM interface also breaks apart when the 3D circuit is stretched (FIG. 13B).

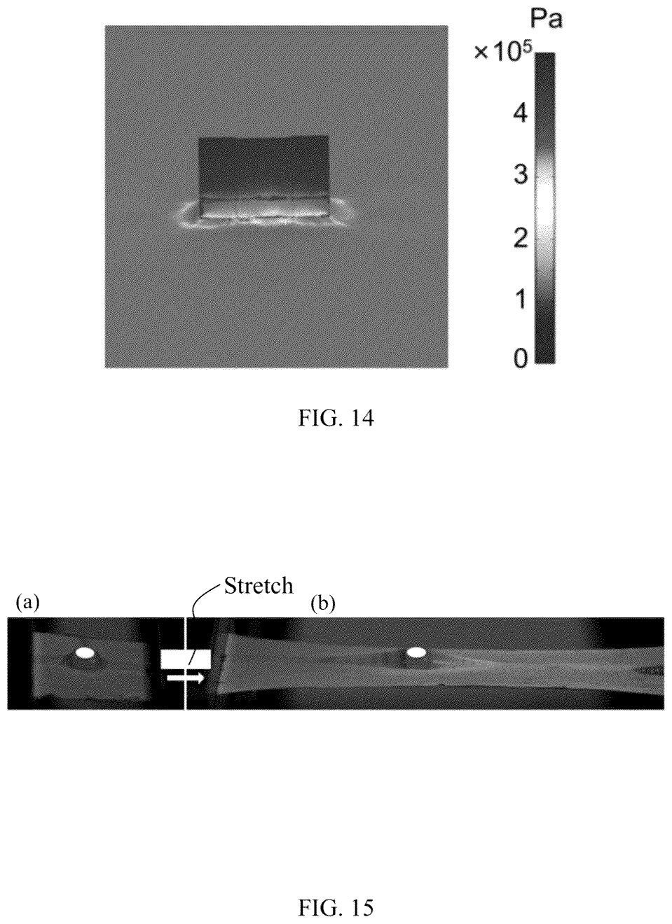

According to one or more embodiments, ultrastretchable hLM solder is developed, which makes use of both the wettability advantage of oLM and the stretchability attribute of LM. As shown in FIG. 13C, additional pristine LM paste is applied at the pin/oLM interface to form the hLM solder (FIG. 13C). The hybrid connection method reduces the stress concentration factor (the ratio of the maximum stress to the average stress, i.e., σₘₐₓ/σ_{avg}.) at the interface between the rigid chip and the soft SBS by 30% (FIG. 13D and FIG. 14), in comparison to those using either single-component pristine LM or oLM as the connection material (see Table 3 below). As a result, the hybrid connection provides outstanding interfacial stability even under large tensile strains. The resistance of a 100Ω rigid microresistor bonded with hLM solder shows negligible change when the circuit is stretched to 1500% strain (FIG. 13E). In contrast, the same circuit using either LM or oLM as solder fails when stretched to less than 50% strain.

TABLE 3
Summary of input parameters and the output results of stress distributions in the
finite element analysis (FEA) of chip interfaces, LM antenna, and Cu antenna.

| | | Average | | | | |
|---|---|---|---|---|---|---|
| | | Young's | Average | | | |
| | | modulus | Poisson's | | | |
| System | Material | (Pa) | ratio | σₘₐₓ (Pa) | σ_{avg} (Pa) | σₘₐₓ/σ_{avg} |
| Chip with LM | SBS | 10424 | 0.47 | 1.25E7 | 9.43E4 | 132.56 |
| or oLM solder | LM/SBS | 9841 | 0.47 | | | |
| | OLM/SBS | 3.1E5 | 0.47 | | | |
| | A1203 for | 3.75E10 | 0.22 | | | |
| | chip | | | | | |
| Chip with hLM | SBS | 10424 | 0.47 | 9.81E6 | 9.41E4 | 104.25 |
| solder | LM/SBS | 9841 | 0.47 | | | |
| | oLM/SBS | 3.1E5 | 0.47 | | | |
| | A1203 for | 3.75E10 | 0.22 | | | |
| | chip | | | | | |
| LM antenna | SBS | 10424 | 0.47 | 8.10E5 | 9.46E4 | 8.56 |
| | LM/SBS | 9841 | 0.47 | | | |
| Cu antenna | SBS | 10424 | 0.47 | 1.15E9 | 2.29E5 | 5021.60 |
| | Cu/SBS | 1.1E11 | 0.34 | | | |

FIG. 13F shows the schematic structure of the 3D circuit using the hLM solder. It should be noted that taking advantage of the good wettability, the oLM spontaneously penetrates through the thin paste mask SBS layer to connect with the underneath LM circuit traces (in this specific case, the upper LM circuit layer). As a consequence, the vertical electrical connection between the pin/oLM and the underneath LM 3D circuit is formed. In the meanwhile, the upper LM circuit layer is connected with the base LM circuit layer using stretchable LM VIAs. As shown in FIGS. 13G and 13H, there is no obvious interfacial gap among the different layers of the P3D-eskin because all fibrous SBS mats are deposited using the electrospinning method. The interfaces remain seamless during stretching or bending deformation.

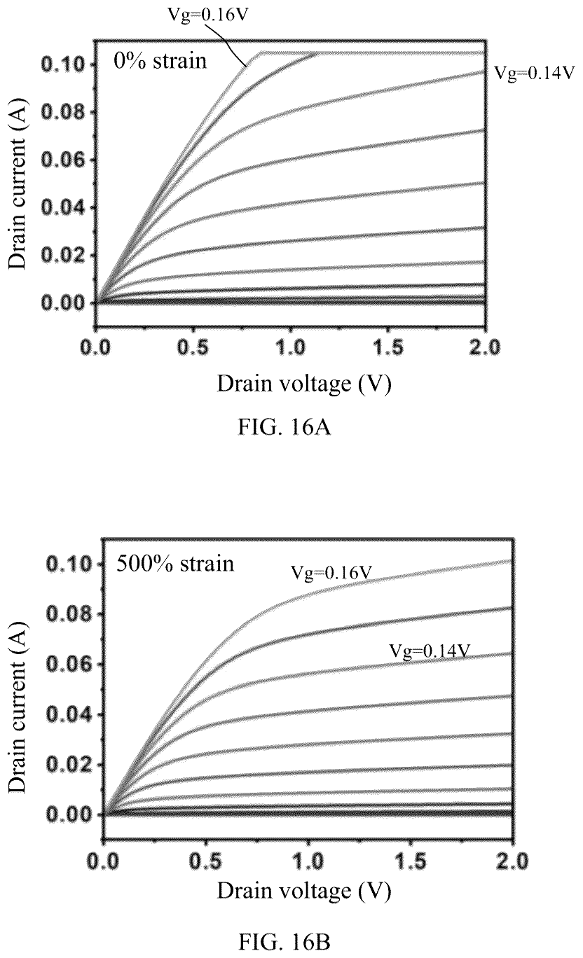

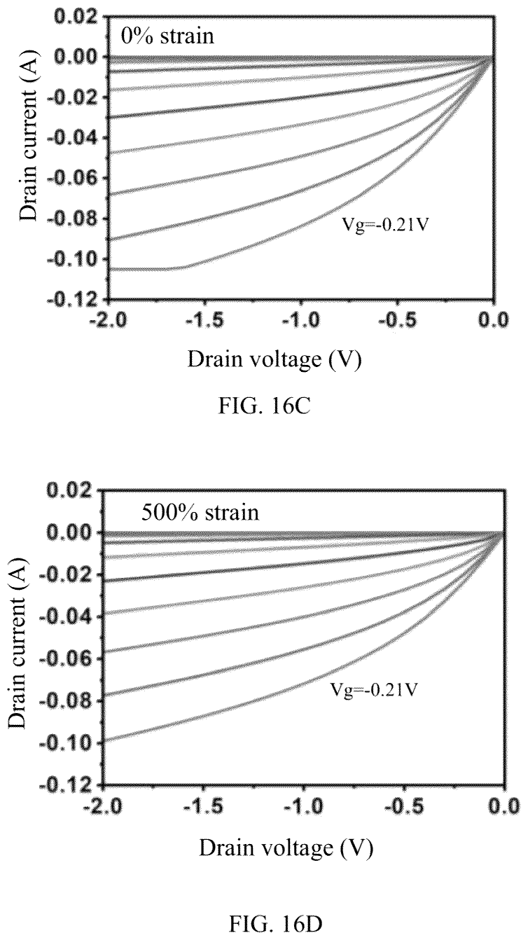

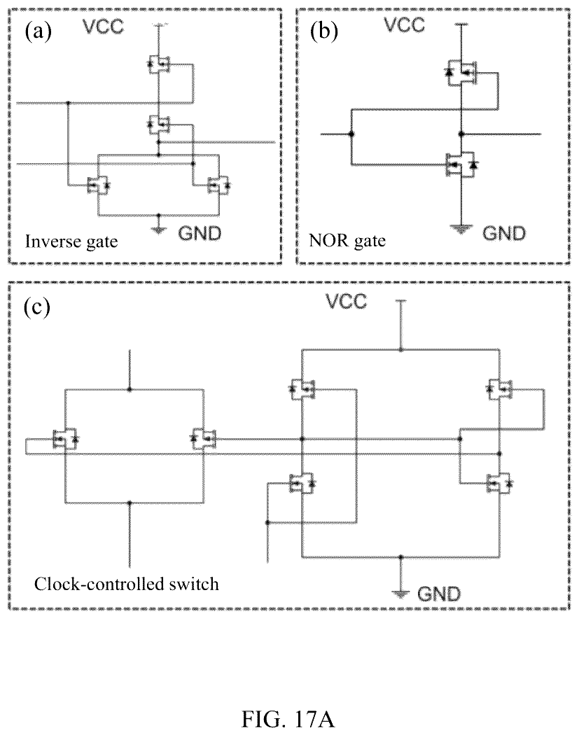

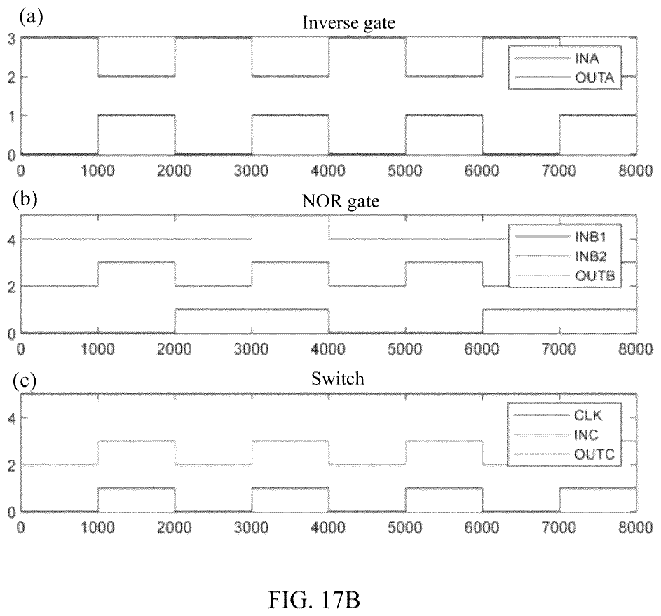

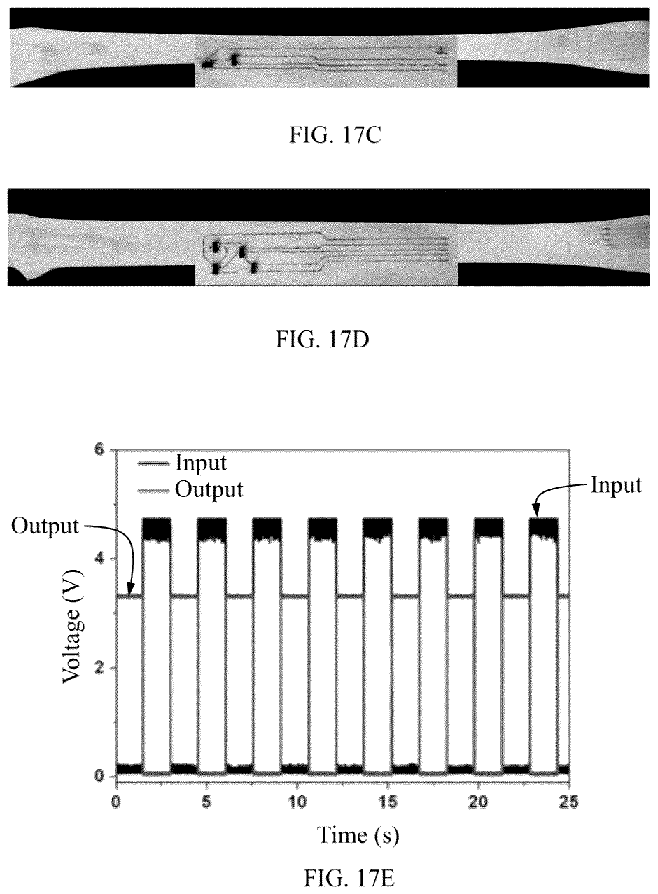

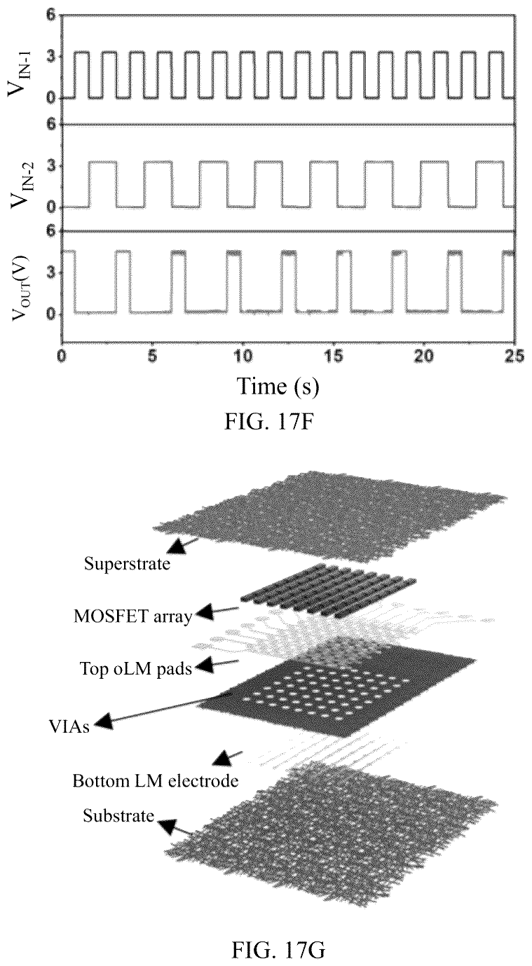

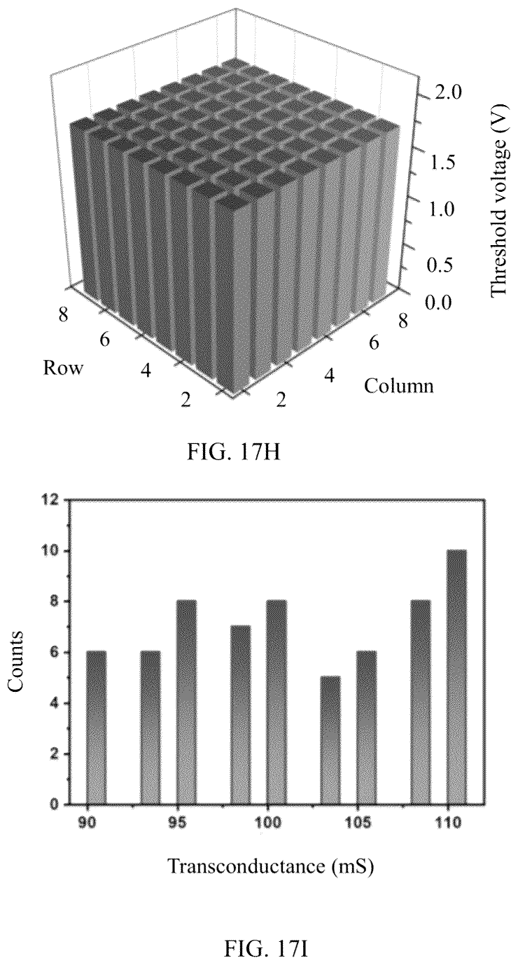

As a proof of the concept of the stable 3D interfaces, different kinds of rigid electronic components, including microresistors, metal-oxide-semiconductor field-effect transistors (MOSFETs) and LEDs to the stretchable 3D LM circuit are tested in terms of their performances under large strains. Connecting to different microresistors ranging from 100Ω to 1 MΩ, the resistance of the circuit shows neglectable change when stretched to 1500% strain (FIG. 13I) and remains stable during the 1000 cycles of stretch-release tests (FIG. 13J). The stable brightness of the LED during the stretching process also indicates the constant resistance of the stretchable circuit (FIG. 15). The stretchable P-type (FIG. 13K and FIG. 16A) and N-type (FIG. 13L and FIG. 16B) MOSFET circuits also shows stable transfer and output characteristics under large strains up to 500%. It is further fabricated stretchable logic circuits including the clock-controlled switch (FIG. 13J and FIG. 13N), inverse gate, NOT-OR (NOR) gate, and 3D switch array with the MOSFETs (FIG. 17A to FIG. 17J). These logic circuits can operate normally in the logic output states under various strains.

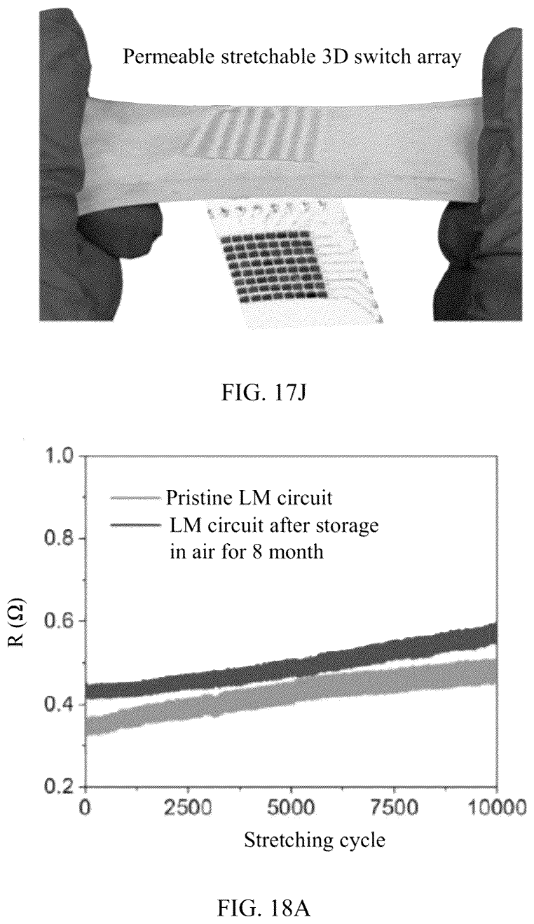

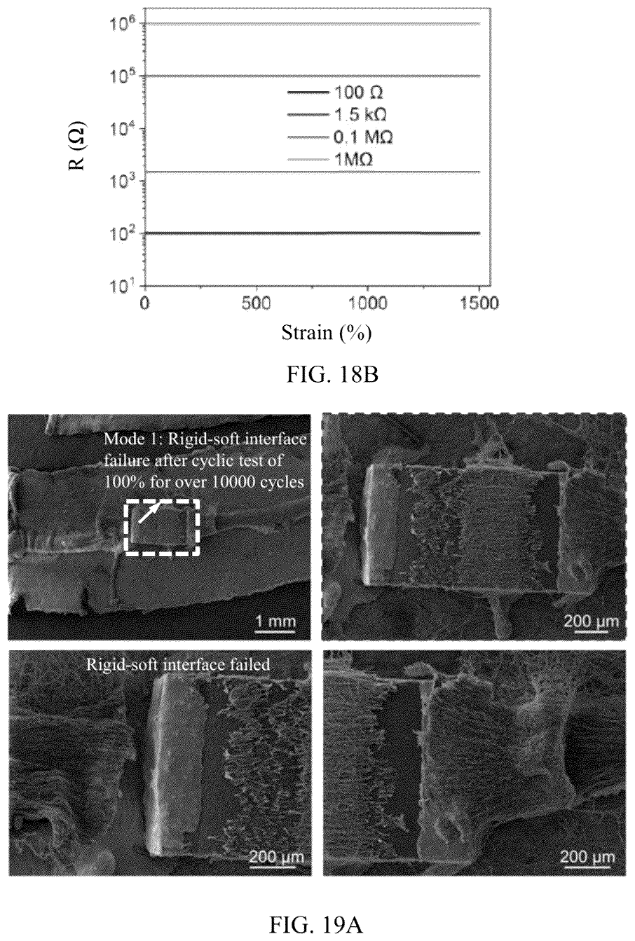

After storing for 8 months, the initial electrical resistance of the LM circuit before cycling increases slightly from 0.33Ω to 0.42Ω. During the stretching tests, samples previously stored in air show similar outstanding electrical stability and robustness. The electrical resistances only increase by 0.119Ω and 0.083Ω for the freshly made sample and the stored sample after the cycling tests, respectively (FIG. 18A). Additionally, the electrical interfaces between LM circuits and the microresistors also possess outstanding stretchability and electrical stability after storage for eight months (FIG. 18B).

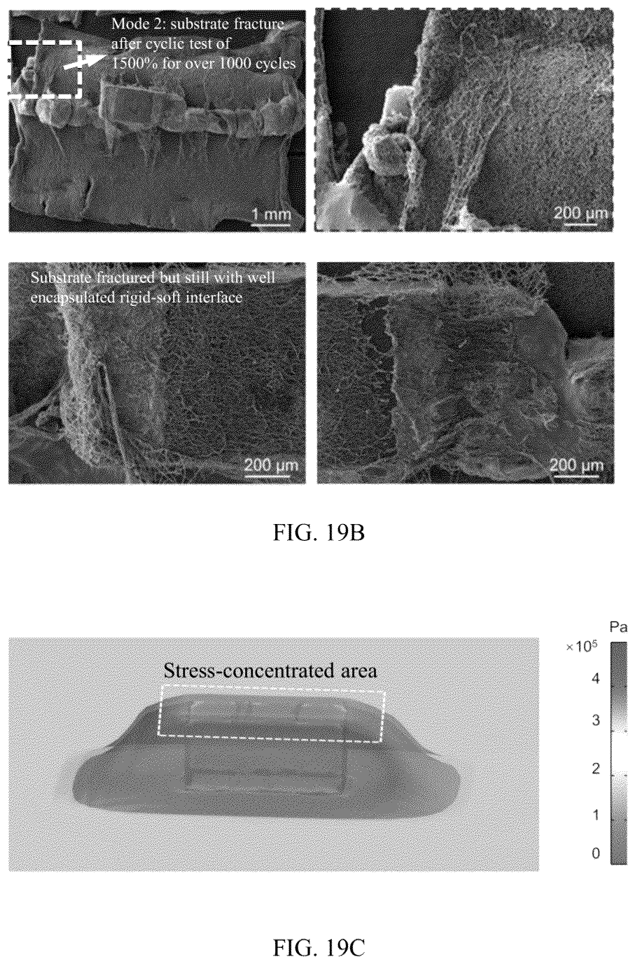

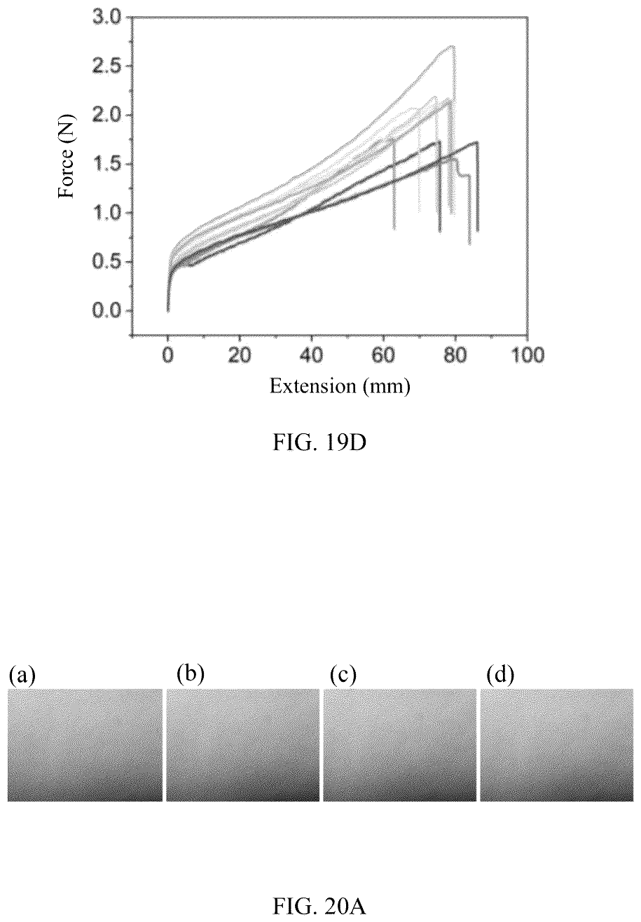

Referring to FIG. 19A to FIG. 19D, the failure modes of the solder joints after long-term repeated cycling tests are discussed. The present inventors have found two typical failure modes of the electrical interfaces that behave differently at low strains (e.g., 100%, FIG. 19A), and high strains (e.g., 1500%, FIG. 19B). At low (e.g., 100%) strain, the interface between the rigid electronic component and the soft encapsulation mat (rigid-soft interface) fails after the sample sustains long cycles (e.g., over 10,000 cycles) of repeated stretch-release process. This is due to the long-term continuous mechanical wearing and tearing of the stress-concentrated rigid-soft interface (FIG. 19C). Finite element analysis (FEA) indicates the maximum stress occurs on the rigid-soft interface between the rigid microchip and the soft SBS fiber mat. At extremely high (e.g., 1500%) strain close to the breaking strain of the substrate material, the substrate fractures after 1,000 cycles of stretch-release tests, while the interface at the solder joins remains well encapsulated. According to the force-extension curves of the SBS mats (n=10), the average breaking force is ˜2.013 N (FIG. 19D), at which the solder joints are still not broken.

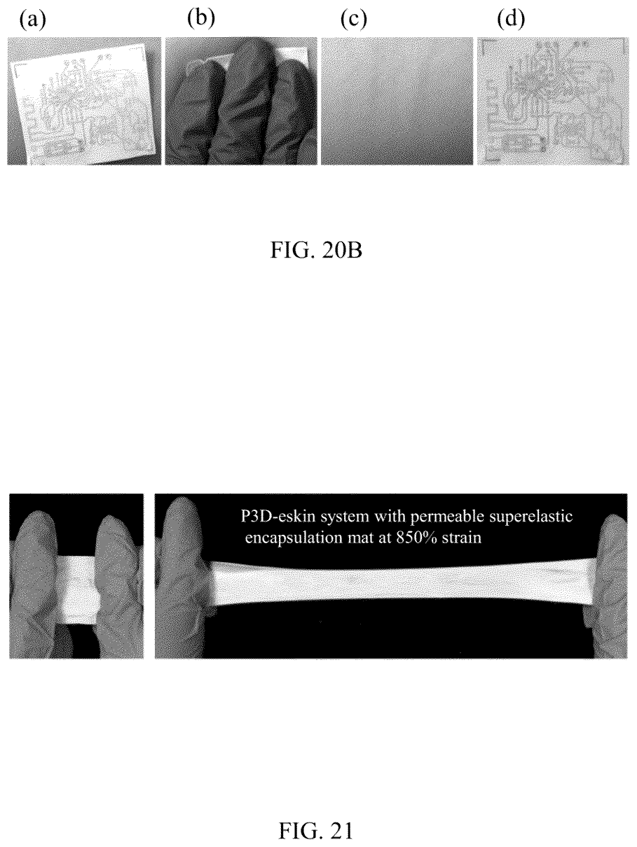

For the leakage concern, when pressing the LM 3D circuits on the arm before mounting the components, the LM 3D circuits do not leak onto the skin even with a high pressure of up to 50 kPa (FIG. 20A). After the on-skin pressing test, the LM 3D circuits maintain intact without any merging of the lines (FIG. 20B). Additionally, the P3D-eskin system with the coverage of the superstrate is still well encapsulated at an extremely large strain of 850% (FIG. 21).

Long-range wireless transcutaneous electrostimulation and electrophysiological sensing system. Wireless transcutaneous electrostimulations and the recording of corresponding electromyography (EMG) signals. All procedures involving the on-skin attachment of P3D-eskins and PDMS-eskins on the human body follow ethical guidelines, which are approved by The Hong Kong Polytechnic University (HSEARS20230101001).

The animal experiments as described herein follow the Ethical Review of Research Experiments Involving Animal Subjects (A-0664) approved by Research Committee (Animal Research Ethics Sub-Committee) of City University of Hong Kong. Prior to transcutaneous electrical stimulations and electrophysiological signal recording, a healthy male Sprague Dawley (SD) rat (aged 4-5 weeks, ˜200 g) is utilized for biceps femoris muscle stimulation. The SD rat is first treated with gaseous light anesthesia (isoflurane, 3%), followed by deep anesthesia by intraperitoneal injection of a mixed solution of ketamine (100 mg/kg) and xylazine (10 mg/kg). The hair on the skin of both legs is shaved for attaching the P3D-eskin patch.

Prior to the on-skin attachment, a biocompatible, soft, and wet-adhesion adhesive is used to adhere the edges of the eskins (PDMS-eskin and the P3D-eskin) onto the skin. The electrostimulation process is wirelessly controlled via a mobile Android system-based mobile phone. The anode and the common cathode are applied to the animal's skin to form a closed circuit. The current control module is connected between the ground (GND) of the circuit and the common cathode, providing a virtual ground (VGND) potential that changes according to the permitted current. The permitted current intensity is controlled by a simple current mirror circuit. The current intensity in the second transistor is identical to the reference current through the first transistor by sharing the same gate (G) and source(S). By sending pre-defined serial commands to the DAC, the voltage is precisely controlled in the range of 0˜3.3 V. This voltage is then applied to the drain (D) and G of the transistor to define the reference current intensity. A fixed resistor (50Ω) is connected in serial to the common D and GND, where the voltage on D reflects the total current. This voltage can be read by the 14-bit analog-digital converter (ADC) on the MCU. To prevent overload voltage from causing damage to MCU, an operational amplifier that functions as a voltage follower (providing identical voltage) is added between D and MCU. Next, the output voltage and current data are measured by a DAQ (data acquisition) multi-meter system (Keithley DAQ6510) at a sampling frequency of 10 kHz. The EMG signals are measured using a high-precision data acquisition system (PowerLab 16/35, AD Instruments) and with a biological signal amplifier (BioAmp FE132, AD Instruments) with a sampling rate of 10 kHz. The raw signal data are digitally filtered by two notch filters at 50 Hz and 100 Hz to obtain the representative EMG waveforms avoiding the baseline noises.

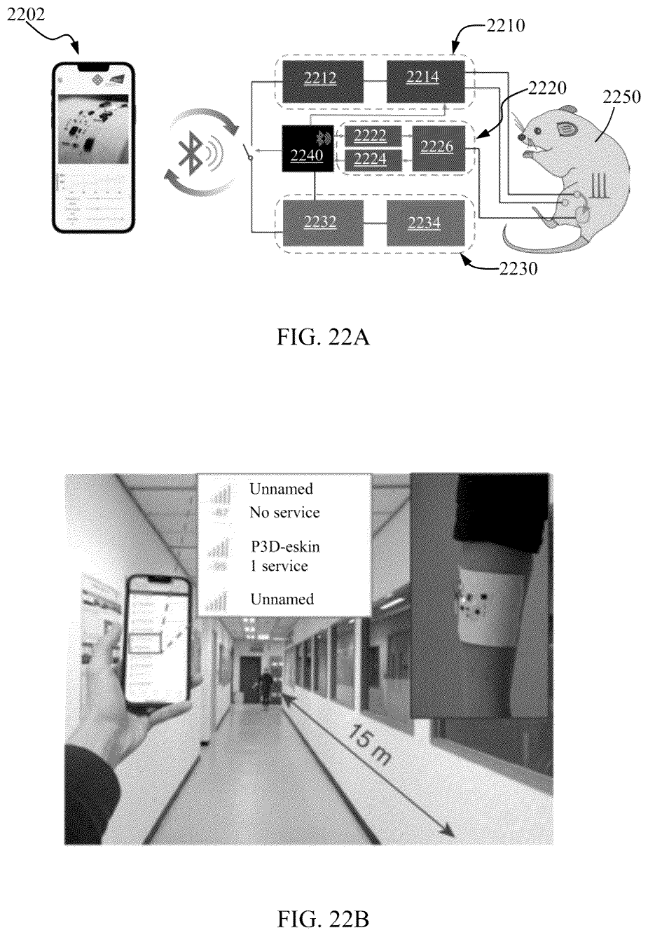

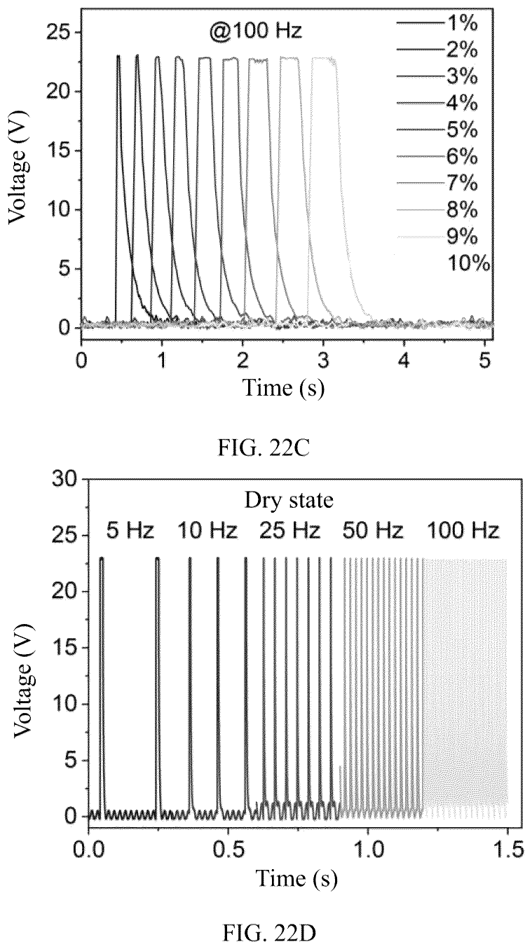

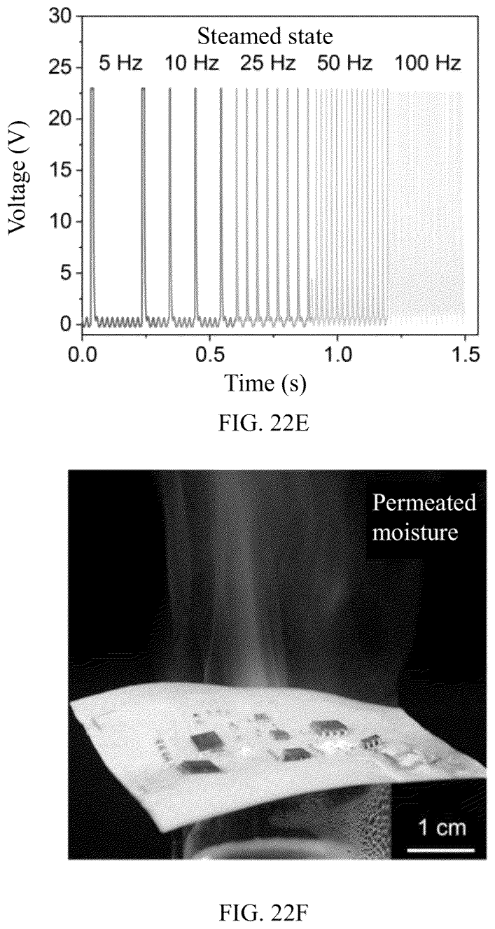

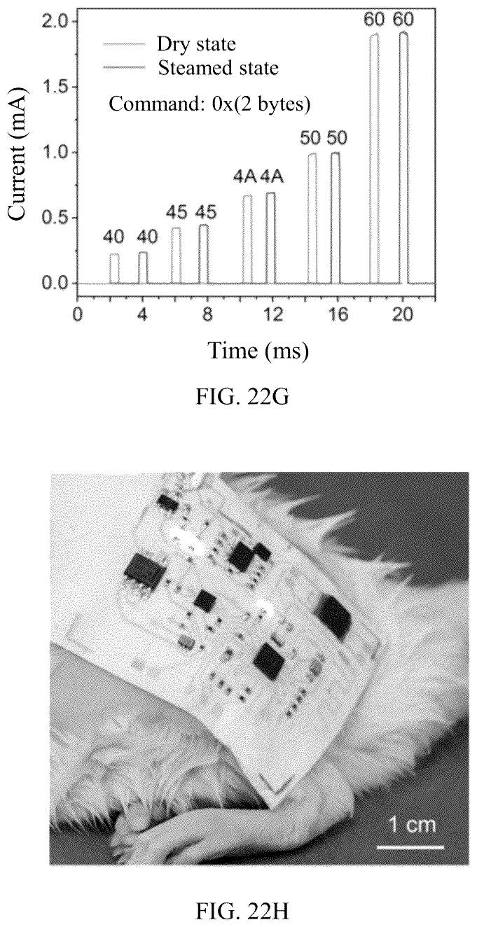

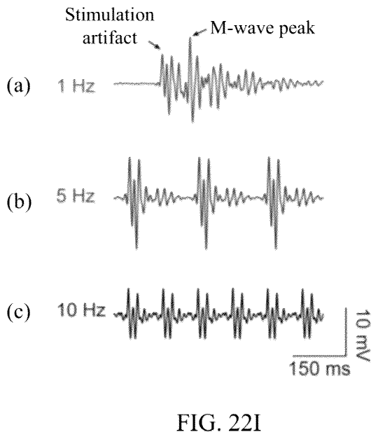

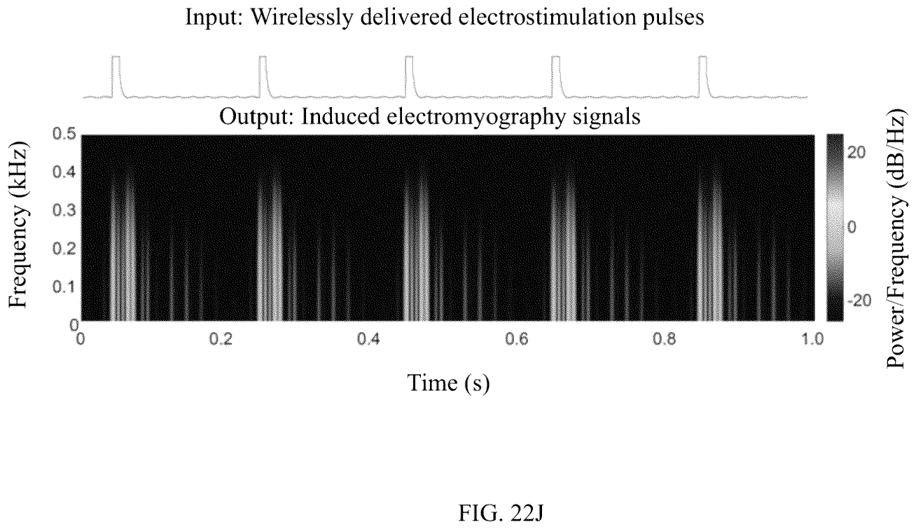

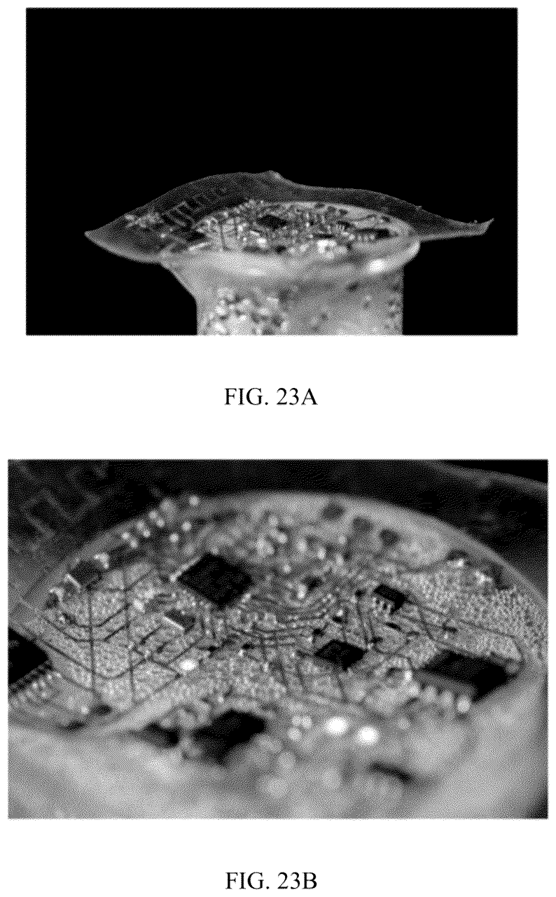

FIG. 22A illustrates the block diagram of a wireless transcutaneous electrostimulation and electrophysiological sensing system fabricated based on the P3D-eskin platform. The diagram illustrates a smartphone 2202 with mobile application or app installed thereon, a stimulation generator 2210, a current control 2220, a power management 2230, a Bluetooth Low Energy (BLE) MCU 2240, and an animal in the form of a rat 2250. The stimulation generator 2210 comprises a 20V booster 2212 and a high voltage (HV) MUX 2214. The current control 2220 comprises a DAC 2222, an OP-AMP 2224 and a current mirror 2226. The power management 2230 comprises a 3.3 V regulator 2232 and a battery 2234. The MCU 3340 is a 5.1 built-in MCU and matched with a 2.4 GHz BLE LM antenna (planar inverted F-shaped antenna), which is capable of providing stable wireless control and data transmission functions with the mobile device at a distance up to 15 m (FIG. 22B). The embedded electro-stimulating electrodes can generate high-voltage electrical pulses with precisely controlled current intensity, frequency, and duty cycle for delivering electrical stimulations to the user/animal's body. By controlling the ON/OFF period of the MUX, the generated DC high voltage is transformed into periodical pulses with precisely controlled frequency (1-100 Hz) and duty cycle (1-10%) (FIG. 22C). Because of the high permeability of P3D-eskin, the generation of electrical pulses does not show any signal drift or electrical failure (FIG. 22D and FIG. 22E) even when the entire P3D-eskin is steamed on top of boiling water (FIG. 22F). In contrast, water droplets accumulate on the surface of the impermeable PDMS-eskin (FIGS. 23A and 23B).

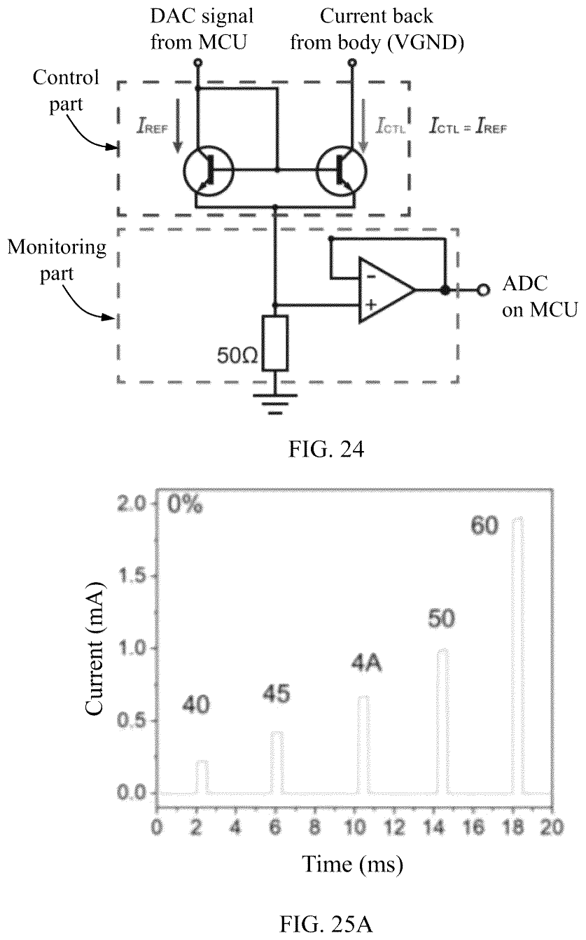

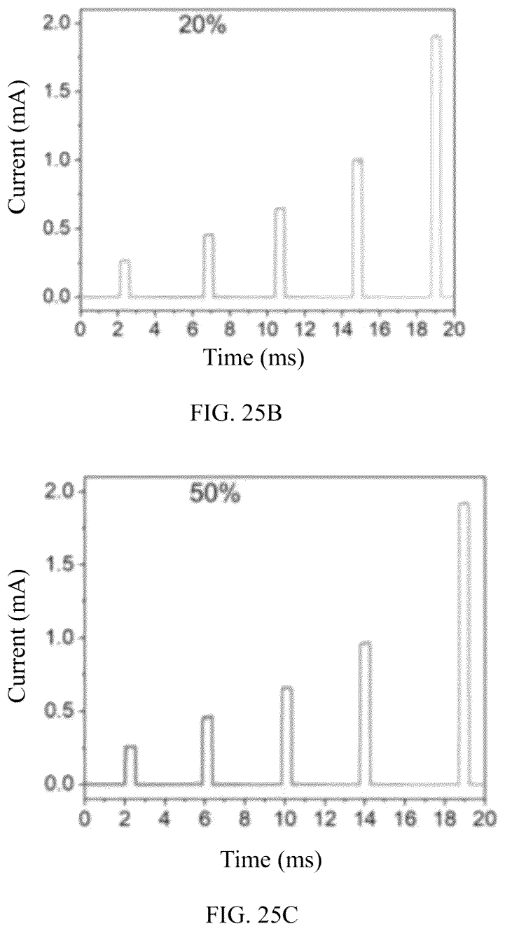

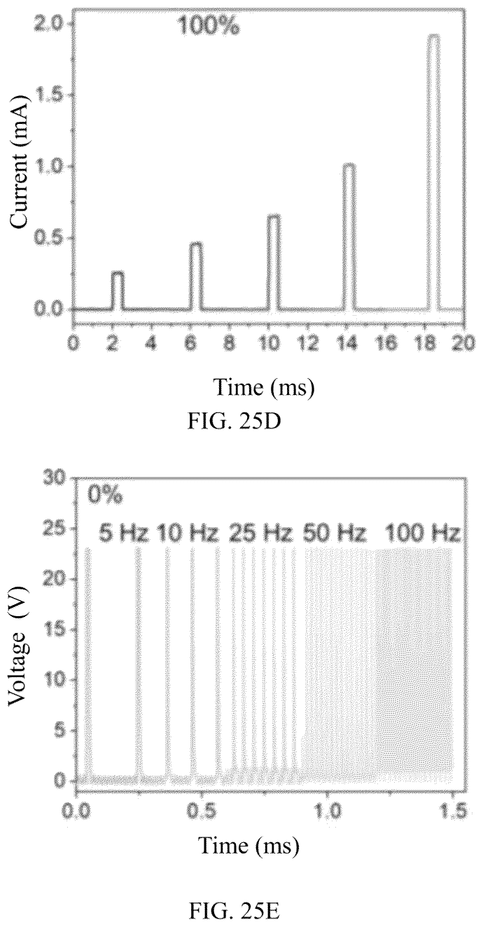

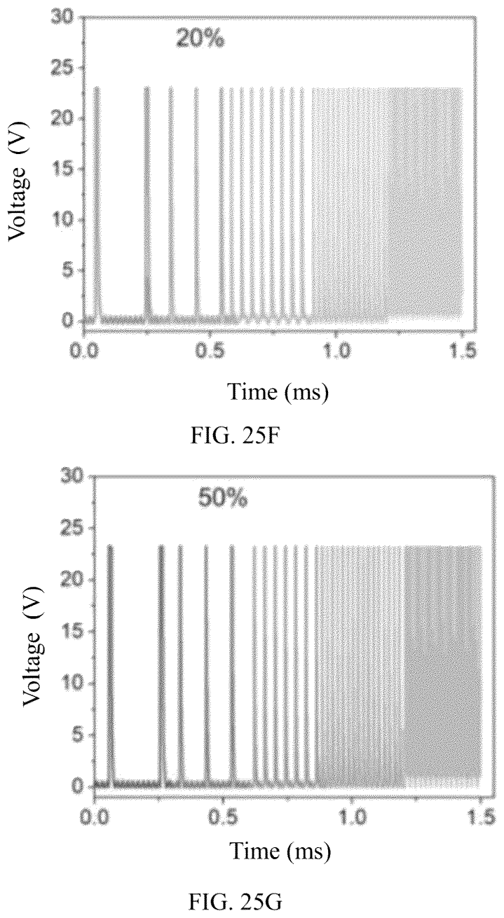

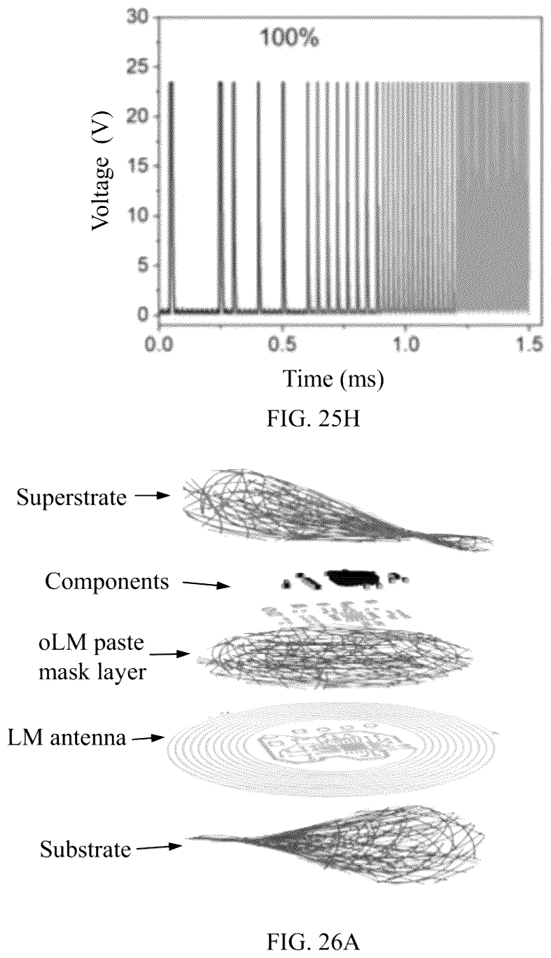

The output voltage of the DAC of the P3D-eskin is controlled by sending the setting commands on the mobile device (FIG. 24). By the skin-interfacing LM electro-stimulating electrodes, the generated current pulses pass through the body and flow into the current mirror of the current control module, where the current intensity is precisely set in the range from 0 to 2 mA (FIG. 22G). The actual current intensity is transformed into a safely sensible voltage for MCU by an OP-AMP. The electrostimulation output waveforms and controls current intensities under stretching states maintain very consistent and stable, demonstrating outstanding system-level stability at stretching states (FIG. 25A to FIG. 25H). This P3D-eskin is used to deliver electrostimulations to the bicep femoris on an experimental animal (FIG. 22H). The real-time electromyography (EMG) signals at adjacent areas on the rat's bicep femoris is recorded. During the stimulation period, corresponding EMG responds to various frequencies (1, 5, and 10 Hz) (FIG. 22I) and matches well with the stimulation input (FIG. 22J), which proves that electrical stimulations are successfully delivered with the wireless P3D-eskin system.

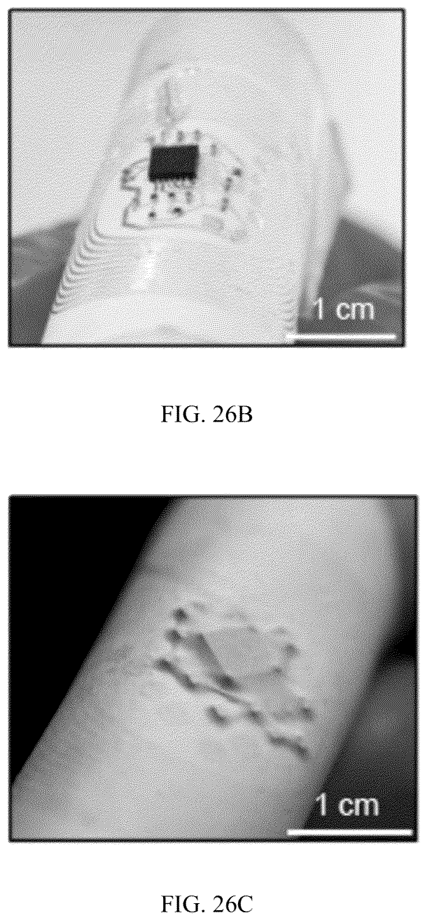

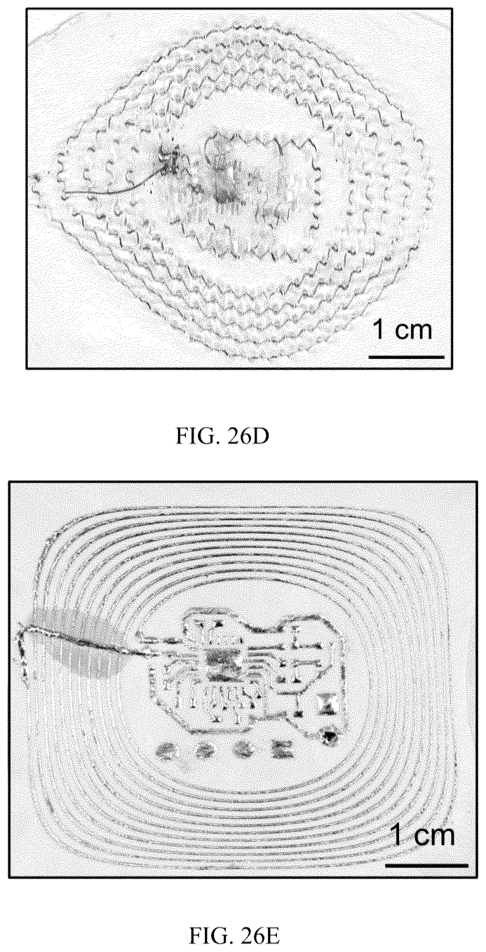

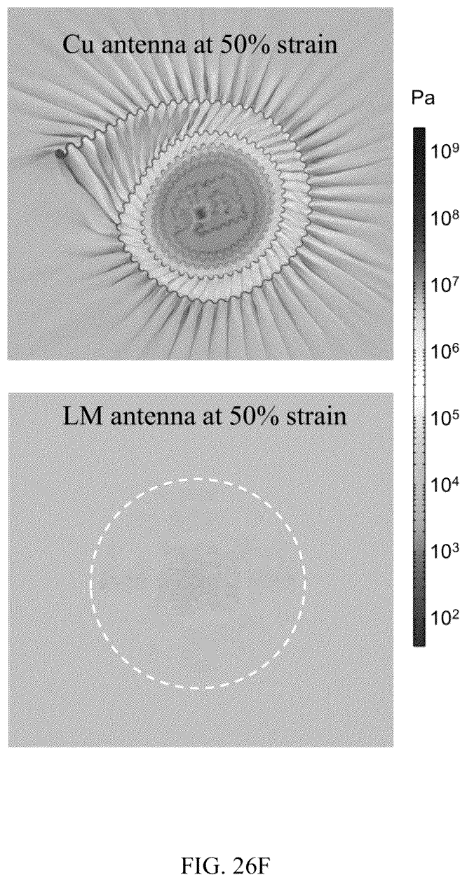

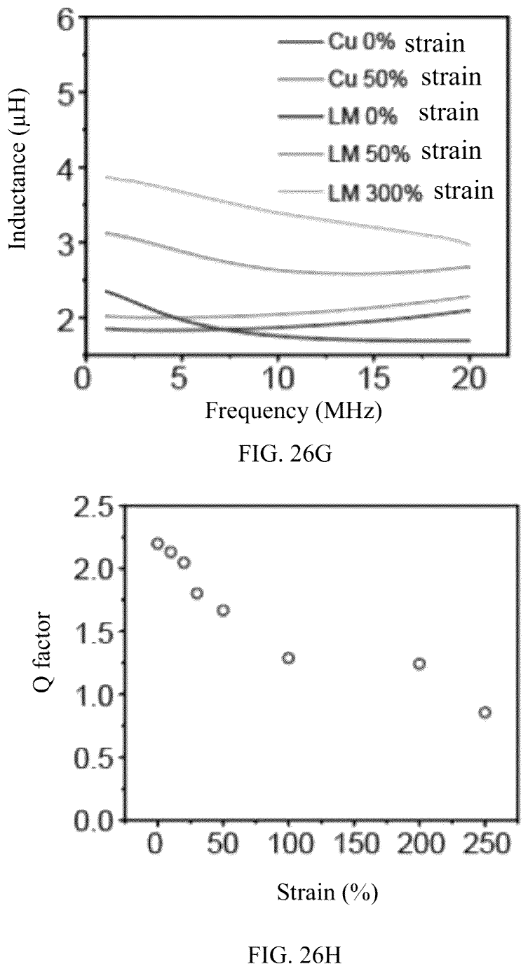

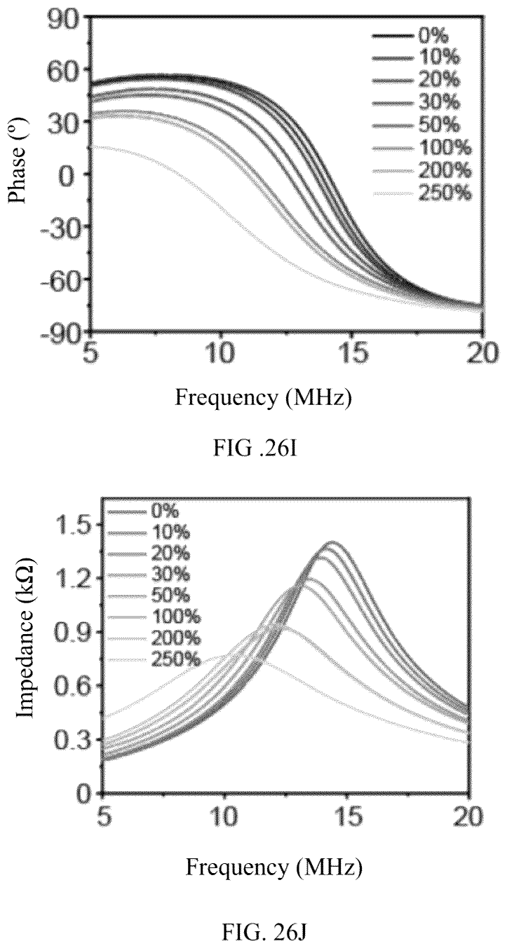

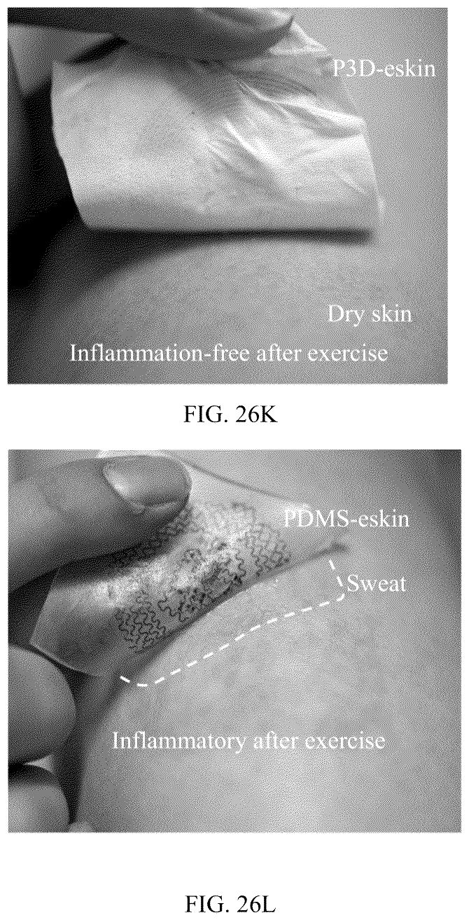

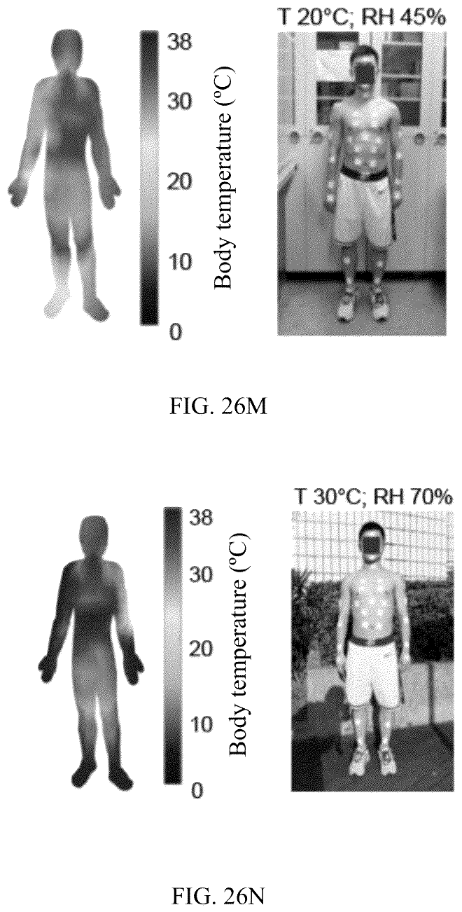

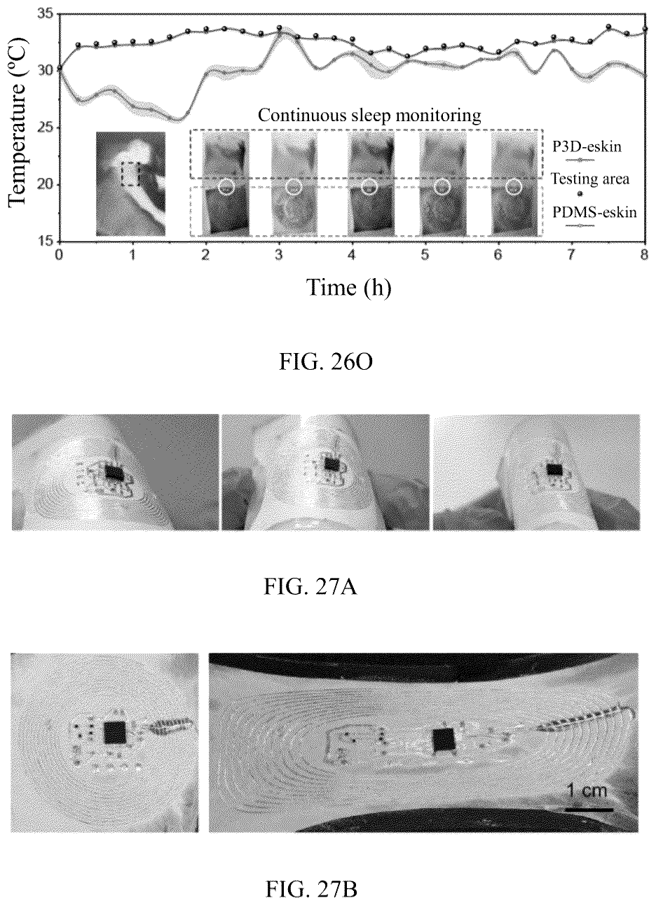

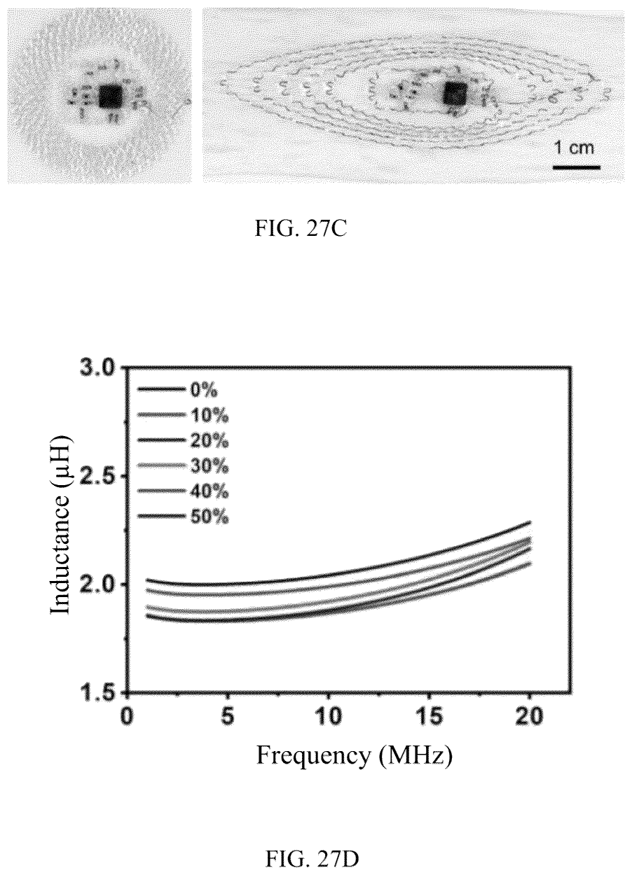

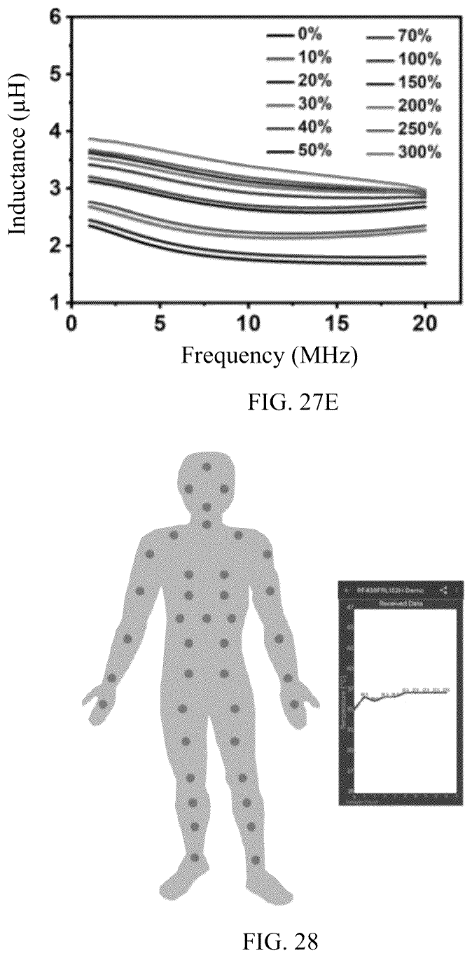

Battery-free P3D-eskin system using near-field communication (NFC). A battery-free type of P3D-eskin is developed using near-field communication (NFC) technology (FIG. 26A). The NFC P3D-eskin before and after the final encapsulation is shown in FIG. 26B and FIG. 26C. It is also highly flexible and stretchable (FIG. 27A).

Characterizations of the near-field communication (NFC) P3D-eskin system. To develop a battery-free type of P3D-eskin, an NFC-embedded MCU (RF430FRL152H, Texas Instruments) is adopted, and the temperature sensing circuit from reference design (TIDM-RF430-TEMPSENSE) is modified. The temperature data are acquired and read by a mobile Android app GUI for temperature sensing (RF430FRL152H Demo, Texas Instruments FIG. 28) or an NFC reader (MSP-EXP430G2ET with TRF7970A NFC/RFID booster pack, FIG. 6). Characteristics of the LM inductive antenna including inductance, Q factor, impedance, and phase are characterized using an impedance analyzer (E4991B, Keysight Technologies).

The LM antenna coils of the NFC outperform those serpentine Cu coils in terms of design compactness, stretchability, and electromagnetic stability. In order to achieve a good stretchability, conventional stretchable Cu antenna coils are fabricated to the shape of serpentine, which significantly reduced the coil density (FIG. 26D). In contrast, due to the intrinsic stretchability of LM, the LM coil is much more compact at the same occupied area. The turn number of the LM coil is twice that of the Cu antenna (FIG. 26E and FIG. 27B). Moreover, the Cu antenna only sustains a stretchability of up to 50% strain, at which the jump wire and the interconnects with the components are disconnected (FIG. 27C), while the LM antenna is well connected even at a strain of 300%.

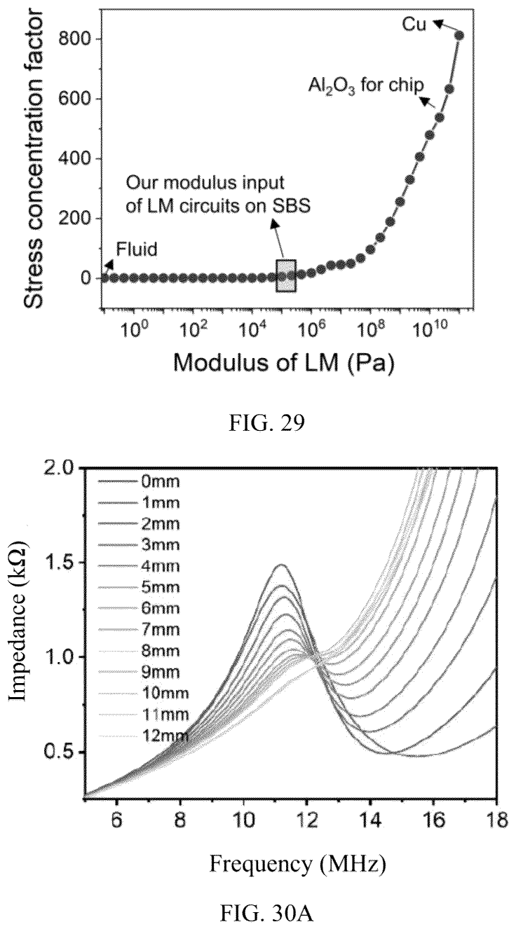

Static structural mechanics of the systems are analyzed using FEA. Material mechanical parameters are summarized in Table 3. The modulus of LM and oLM is measured from the stress-strain curves. Since LM or oLM is neither self-supporting material nor pure liquid (with the inevitable existence of an oxide layer), Young's modulus of LM or oLM supported with the substrate (SBS fiber mat) is tested. Accordingly, Young's modulus values of LM traces (LM on the SBS fiber mats) are adopted for FEA of the stress distribution of the electrical interfaces. A sensitivity test is conducted by varying the modulus of LM ranging from 0.1 Pa (fluid-like substance) to 1011 Pa (near the modulus of Cu) and the change of stress concentration factor (the ratio of the maximum stress to the average stress) is observed. The modulus of LM shows little influence on the stress concentration factor within the modulus range of 0.1 Pa to 1 MPa, of which all these stress concentration factors are much smaller than those in the controlled groups of simulations (e.g., Al₂O₃ for chip and Cu antenna), as shown in FIG. 29. Four systems are investigated, i.e., serpentine Cu antenna, intrinsically stretchable LM antenna, a chip with LM or oLM solder, and a chip with hLM solder. These systems are subjected to mechanical tension of 50% strain. The stress responses are collected. The ratio of the maximum stress to the average stress is used as the stress concentration factor, i.e., σₘₐₓ/σ_{avg}. The input parameters and output results of FEA are summarized in the above Table 3.

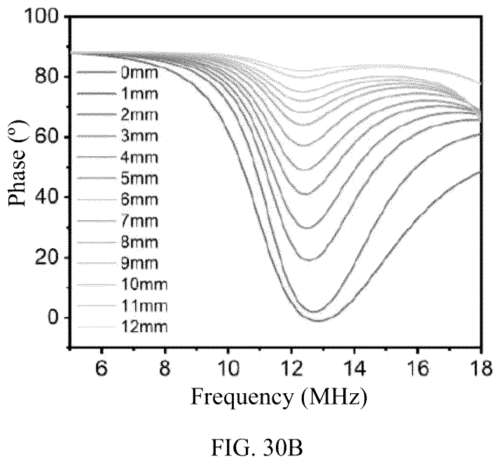

FEA results indicate that this is because the stress distribution of the LM antenna is much more uniform than that of the Cu antenna. The stress concentration factor of the LM coil is only 1/586 of the Cu coil (FIG. 26F, Table 3). Furthermore, the LM coil shows higher inductance than the Cu coil (FIG. 26G, FIG. 27D and FIG. 27E). The Q factor (FIG. 27H), phase (FIG. 27I), and impedance (FIG. 27J) of the LM coil pertain to high stability under various tensile strains and working distances (FIG. 30A and FIG. 30B) within the readable frequency of ˜13.56 MHz.

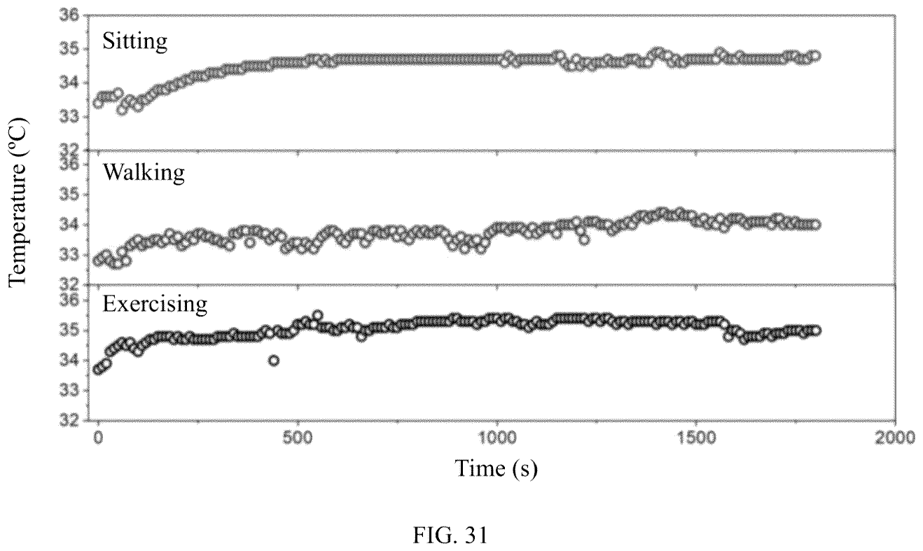

A temperature-sensing NFC P3D-eskin is developed to continuously record the temperature distribution of different positions of the human body (FIG. 28). Continuous monitoring of body temperature during other daily activities such as sitting, walking, and exercising could be stably monitored using a customized mobile application (FIG. 31). P3D-eskins offers a high degree of wearing comfort and biocompatibility to skin health. Wearing the P3D-eskin during intensive exercise does not lead to the accumulation of sweat, thus avoiding skin dampness, allergy, and inflammation (FIG. 26K), while the skin covered by the PDMS-eskin shows obvious skin erythema (FIG. 26L).

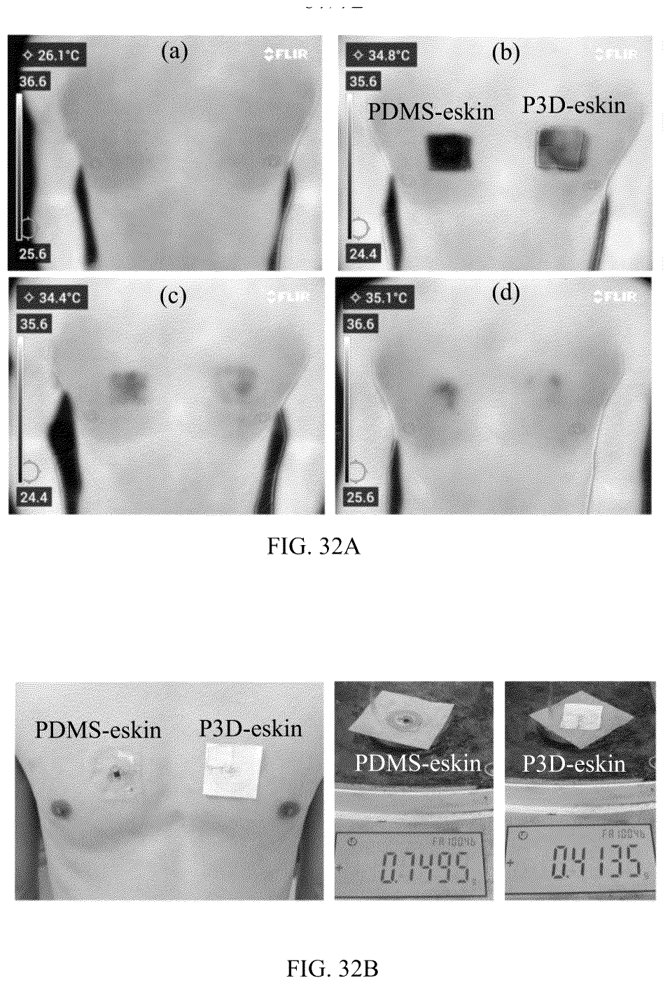

Thermal imagery of an adult male body in cool/dry (FIG. 26M) and hot/humid (FIG. 26N) environments can be depicted by the multi-position physiological temperature mapping with forty NFC P3D-eskin arrays. The body temperature is recorded during continuous sleep monitoring for 8 h (FIG. 26O). Importantly, not only the P3D-eskin shows a more stable signal recording with lower signal variations than PDMS-eskin, but also the recorded temperature values from the P3D-eskin are in a good accordance with the standard temperature recorded with a commercial infrared thermal imager. In the testing conditions, P3D-eskin possesses an initial smaller cooling load, making a smaller thermal impact on the skin than the PDMS-eskin (FIG. 32A and FIG. 32B). Additionally, the permeable characteristic of P3D-eskin allows better convective heat transfer between air and the skin, which is closer to the real situation where bare skin can regulate the body temperature in comparison to the unbreathable dressing with heat conduction only. Further, the P3D-eskin is softer and more stretchable, allowing a more conformal contact with the skin, and may thus reduce the thermal artifacts with smaller signal fluctuations.

According to one or more embodiments, permeable bioelectronics incorporating commercially available electronic components such as high-performance and inexpensive chips with stretchable printed circuits can potentially provide clinical-quality and continuous health monitoring and modulations, beyond the confines of traditional hospitals or laboratories. For long-term wearing ability, permeable and stretchable type of 3D integrated electronics can endow chronic wearing comfort and health in comparison to conventional impermeable thin-film counterparts.

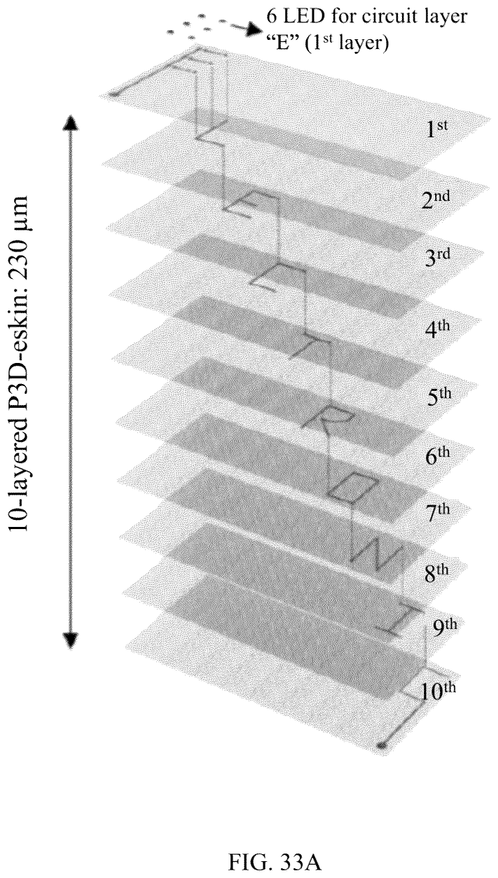

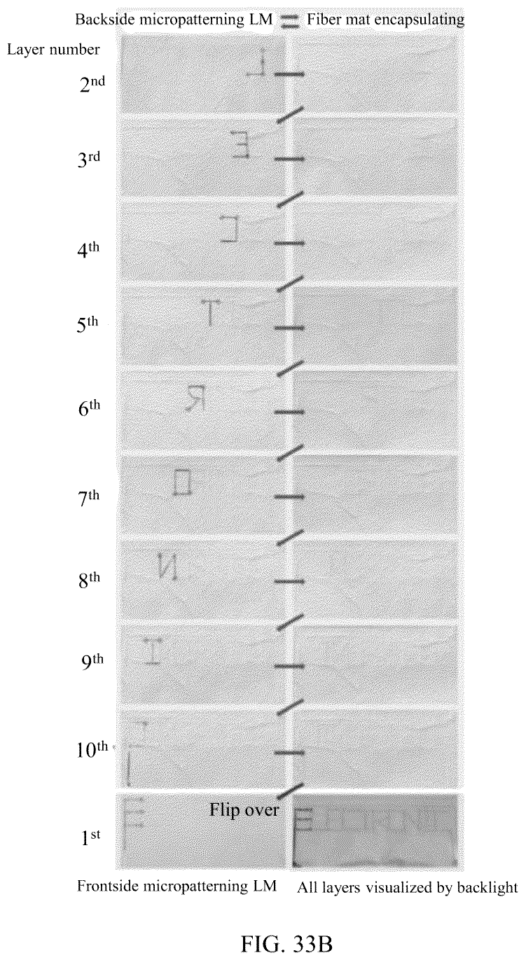

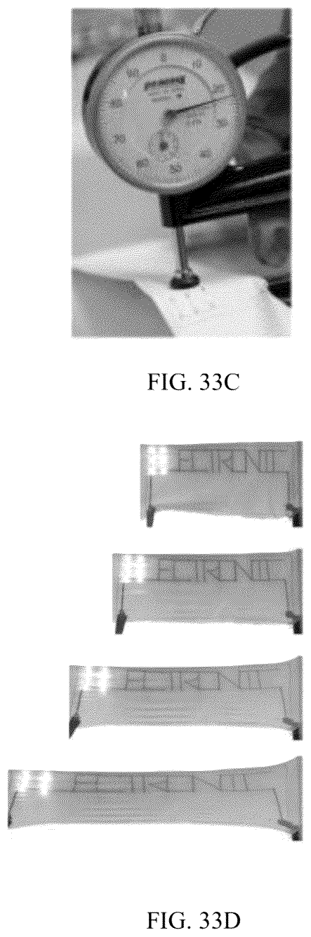

The P3D-eskin platform as described herein according to one or more embodiments offers the first demonstration of how to achieve 3D integrated circuit board with unique advantages of high-density functionalities, skin-like softness and stretchability, and high permeability to air and moisture. The ultrastretchable hLM solder provides a reliable and scalable solution to integrating well-developed rigid electronic components with stretchable fibrous substrates in a 3D configuration. The P3D-eskin integration strategy provides the possibility of fabricating thin, soft, and stretchable multilayered circuits by in situ electrospinning fiber mat and micropatterning LM circuit repeatedly (FIG. 33A to FIG. 33C). In comparison to similar eskins made with stretchable thin film substrates, the P3D-eskin as described herein according to one or more embodiments is significantly lighter, thinner, softer, more stretchable, and most importantly, offers chronic biocompatibility in the on-skin test. P3D-eskin also outperforms prior art permeable electronics in terms of its remarkable system-level high-density integration, and the needlessness to use external PCBs.

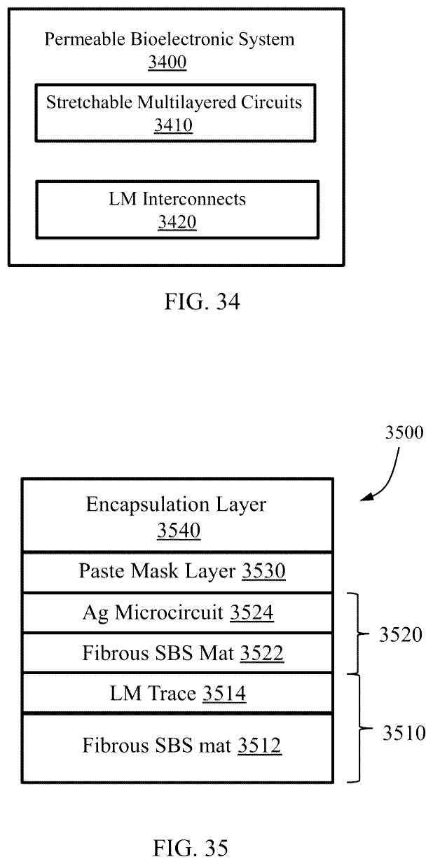

FIG. 34 illustrate a permeable bioelectronic system 3400 according to certain embodiments of the present disclosure. The permeable bioelectronic system 3400 may be one or more systems as described above with reference to one or more embodiments and/or as shown in one or more figures.

As illustrated, the permeable bioelectronic system 3400 comprises stretchable multilayered circuits 3410 comprising liquid metal (LM), and LM interconnects 3420 for bonding one or more inorganic electronic circuit components to the stretchable multilayered circuits. The stretchable multilayered circuits 3410 or the LM interconnects 3420 may be one or more circuits or LM interconnects as described above with reference to one or more embodiments and/or as shown in one or more figures, such as FIG. 1A.

FIG. 35 illustrates a permeable bioelectronic system 3500 according to certain embodiments of the present disclosure. The permeable bioelectronic system 3500 may be one or more systems as described above with reference to one or more embodiments and/or as shown in one or more figures.

The permeable bioelectronic system 3500 comprises a first LM circuit layer 3510 that comprises micropatterned LM. The first LM circuit layer 3510 may be one or more layers as described above with reference to one or more embodiments. For example, the first LM circuit layer 3510 may be the base circuit layer as described above. The first LM circuit layer 3510 may comprise a fibrous SBS mat 3512 and LM trace 3514 formed on the fibrous SBS mat 3512.

The permeable bioelectronic system 3500 further comprises a second LM circuit layer 3520. The second LM circuit layer 3520 comprises micropatterned LM and is formed on the first LM circuit layer 3510. The second LM circuit layer 3520 may be one or more layers as described above with reference to one or more embodiments. For example, the second LM circuit layer 3520 may be the upper circuit layer as described above. The second LM circuit layer 3520 may comprise a fibrous SBS mat 3522 formed on the first LM circuit layer 3510 and a microcircuit in the form of Ag microcircuit 3524 formed on the fibrous SBS mat 3522.

The permeable bioelectronic system 3500 further comprises a paste mask layer 3530 formed on the second LM circuit layer 3520. The paste mask layer 3530 is configured to bond the one or more inorganic electronic circuit components.

The permeable bioelectronic system 3500 further comprises an encapsulation layer 3540 formed on the paste mask layer 3530 for encapsulating the first LM circuit layer 3510, the second LM circuit layer 3520, and the paste mask layer 3530. The encapsulation layer 3540 may comprise fibrous SBS mats.

By way of example, the paste mask layer 3530 may comprise or be made of a fibrous SBS mat (called second fibrous SBS mat) such that the Ag microcircuit 3524 is sandwiched between the second fibrous SBS mat and the fibrous SBS mat 3522 (called first fibrous SBS mate). The first fibrous SBS mat is thicker than the second fibrous SBS mat. For example, the first fibrous SBS mat may have a thickness of 100 μm. The first fibrous SBS mat may have a thickness of 30 μm.

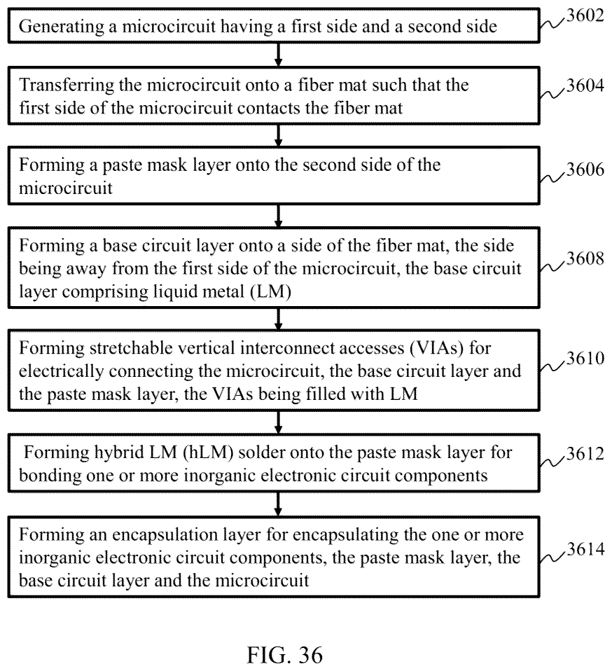

FIG. 36 illustrates a method for fabricating a permeable bioelectronic system according to certain embodiments of the present disclosure. The method may be performed by one or more systems as described above with reference to one or more embodiments and/or as shown in one or more figures.

Block 3602 states generating a microcircuit having a first side and a second side. The microcircuit may be an Ag microcircuit. The first and second sides may be the side of the Ag microcircuit as shown in FIG. 2A. The first side may be the lower side of the Ag microcircuit 3524 as shown in FIG. 35 where the lower side is at the interface between the Ag microcircuit 3524 and the fibrous SBS mat 3522. The second side may be the upper side of the Ag microcircuit 3524 as shown in FIG. 35 where the upper side is at the interface between the Ag microcircuit 3524 and the paste mask layer 3530.

Block 3604 states transferring the microcircuit onto a fiber mat such that the first side of the microcircuit contacts the fiber mat. This step may comprise preparing a polymer solution by dissolving styrene-butadiene-styrene (SBS) polymer in a mixed solvent of tetrahydrofuran and dimethylformamide, and electrospinning the polymer solution onto the microcircuit.

Block 3606 states forming a paste mask layer onto the second side of the microcircuit. This may comprise selectively wetting the microcircuit by using LM and electrospinning SBS mat onto the microcircuit.

Block 3608 states forming a base circuit layer onto a side of the fiber mat, the side being away from the first side of the microcircuit, the base circuit layer comprising liquid metal (LM). This may comprise stencil printing LM traces onto the side of the fiber mat and electrospinning SBS mat onto the LM traces-printed side of the fiber mat.

Block 3610 states forming stretchable vertical interconnect accesses (VIAs) for electrically connecting the microcircuit, the base circuit layer and the paste mask layer, the VIAs being filled with LM. This may comprise creating through holes by laser cutting the microcircuit, the base circuit layer and the paste mask layer and filling the through holes with LM.

Block 3612 states forming hybrid LM (hLM) solder onto the paste mask layer for bonding one or more inorganic electronic circuit components. Block 3614 states forming an encapsulation layer for encapsulating the one or more inorganic electronic circuit components, the paste mask layer, the base circuit layer and the microcircuit.

In one or more embodiments as described above, the SBS fiber mat is used. It should be understood by those skilled in the art that this is for illustrative purpose only. In one or more embodiments, the fiber mats may comprise a styrene-isoprene-styrene block copolymer, a styrene-polybutadiene-styrene block copolymer, a styrene-butadiene block copolymer, a poly(styrene-block-butadiene-block-styrene) copolymer, a polyisoprene rubber, a butadiene rubber, a polyurethane, a thermoplastic polyurethane, a polyvinyl alcohol, a polycaprolactone, polycaprolactone or a mixture thereof. In certain embodiments, the biocompatible elastomeric fiber comprises poly(styrene-block-butadiene-block-styrene).

As used herein, the term “electronic skin” or “eskin” refers to soft and stretchable electronics that mimic the functionalities of human or animal skin.

As used herein, the terms “fibrous mat” and “fiber mat” are used interchangeably to refer to a fiber-based hierarchical porous woven or textile. The fibrous mat may be used as various layers, such as a substrate, an interlayer, an encapsulation layer, or any other proper layer in a bioelectronic system or device or electronics.

As used herein, the term “fiber” refers to an elongated, slender, thread-like and/or filamentous structure.

As used herein, the terms “flexible”, “bendable”, or “stretchable” refer to the ability of a material, structure, device or device component to be deformed into a curved or bent shape or stretched without undergoing a transformation that introduces significant strain, such as strain characterizing the failure point of a material, structure, device or device component, such that it remains intact during bending, folding, or stretching.

As used herein, the term “rigid” refers to the ability of a material, structure, device or device component that is not flexible, not bendable, or not stretchable.

It will further be appreciated that any of the features in the above embodiments of the disclosure may be combined together and are not necessarily applied in isolation from each other. Similar combinations of two or more features from the above described embodiments or preferred forms of the disclosure can be readily made by one skilled in the art.

Unless otherwise defined, the technical and scientific terms used herein have the plain meanings as commonly understood by those skill in the art to which the example embodiments pertain. Embodiments are illustrated in non-limiting examples. Based on the above disclosed embodiments, various modifications that can be conceived of by those skilled in the art would fall within spirits of the example embodiments.

What is claimed is:

1. A permeable bioelectronic system comprising:
stretchable multilayered circuits comprising liquid metal (LM); and
LM interconnects for bonding one or more inorganic electronic circuit components to the stretchable multilayered circuits.

2. The permeable bioelectronic system of claim 1, wherein the LM comprises gallium, a gallium alloy, or a mixture thereof.

3. The permeable bioelectronic system of claim 1, wherein the LM comprises eutectic gallium indium alloy (EGaIn), gallium indium tin alloy (GaInSn), or a mixture thereof.

4. The permeable bioelectronic system of claim 1, wherein the LM interconnects comprise hybrid LM (hLM) solder.

5. The permeable bioelectronic system of claim 4, wherein the hLM solder comprises a combination of partially oxidized LM (oLM) and LM.

6. The permeable bioelectronic system of claim 4, wherein the hLM solder comprises eutectic gallium indium alloy (EGaIn).

7. The permeable bioelectronic system of claim 1, wherein the stretchable multilayered circuits comprise a plurality of layers, and one or more of the plurality of layers comprises fiber mats.

8. The permeable bioelectronic system of claim 7, wherein the fiber mats comprise a styrene-isoprene-styrene block copolymer, a styrene-polybutadiene-styrene block copolymer, a styrene-butadiene block copolymer, a poly(styrene-block-butadiene-block-styrene) copolymer, a polyisoprene rubber, a butadiene rubber, a polyurethane, a thermoplastic polyurethane, a polyvinyl alcohol, a polycaprolactone, polycaprolactone, or a mixture thereof.

9. The permeable bioelectronic system of claim 1, wherein the stretchable multilayered circuits comprise a plurality of layers that are electrically connected by stretchable vertical interconnect accesses (VIAs) filled with LM.

10. The permeable bioelectronic system of claim 1, wherein the stretchable multilayered circuits comprise:
a first LM circuit layer comprising micropatterned LM;
a second LM circuit layer comprising micropatterned LM and formed on the first LM circuit layer; and
a paste mask layer formed on the second LM circuit layer and configured to bond the one or more inorganic electronic circuit components.

11. The permeable bioelectronic system of claim 10, wherein each of the first LM circuit layer and the paste mask layer comprises fibrous styrene-butadiene-styrene (SBS) mats.

12. The permeable bioelectronic system of claim 10, wherein the stretchable multilayered circuits further comprise an encapsulation layer formed on the paste mask layer for encapsulating the first LM circuit layer, the second LM circuit layer, and the paste mask layer.

13. The permeable bioelectronic system of claim 12, wherein the encapsulation layer comprises fibrous styrene-butadiene-styrene (SBS) mats.

14. The permeable bioelectronic system of claim 10, wherein the stretchable multilayered circuits comprise a silver (Ag) circuit sandwiched between a first fibrous styrene-butadiene-styrene (SBS) mat and a second fibrous SBS mate,
wherein the first fibrous SBS mat is positioned between the first LM circuit layer and the second fibrous SBS mate, and the first fibrous SBS mat is thicker than the second fibrous SBS mat.

15. The permeable bioelectronic system of claim 1, wherein the stretchable multilayered circuits comprise a Near Field Communication (NFC)-embedded microcontroller unit.

16. A method for making a permeable bioelectronic system, the method comprising:
generating a microcircuit having a first side and a second side;
transferring the microcircuit onto a fiber mat such that the first side of the microcircuit contacts the fiber mat;
forming a paste mask layer onto the second side of the microcircuit;
forming a base circuit layer onto a side of the fiber mat, the side being away from the first side of the microcircuit, the base circuit layer comprising liquid metal (LM);
forming stretchable vertical interconnect accesses (VIAs) for electrically connecting the microcircuit, the base circuit layer and the paste mask layer, the VIAs being filled with LM;
forming hybrid LM (hLM) solder onto the paste mask layer for bonding one or more inorganic electronic circuit components; and
forming an encapsulation layer for encapsulating the one or more inorganic electronic circuit components, the paste mask layer, the base circuit layer and the microcircuit.

17. The method of claim 16, wherein the step of transferring the microcircuit onto the fiber mat comprises:
preparing a polymer solution by dissolving styrene-butadiene-styrene (SBS) polymer in a mixed solvent of tetrahydrofuran and dimethylformamide; and
electrospinning the polymer solution onto the microcircuit.

18. The method of claim 16, wherein the step of forming the paste mask layer comprises:
selectively wetting the microcircuit by using LM; and
electrospinning SBS mat onto the microcircuit.

19. The method of claim 16, wherein the step of forming the base circuit layer comprises:
stencil printing LM traces onto the side of the fiber mat; and
electrospinning SBS mat onto the LM traces-printed side of the fiber mat.

20. The method of claim 16, wherein the step of forming the VIAs comprises:
creating through holes by laser cutting the microcircuit, the base circuit layer and the paste mask layer; and
filling the through holes with LM.
